(19) United States
(12) Patent Application Publication
(10) Pub. No.: US 2006/0141614 A1
(43) Pub. Date: Jun. 29, 2006

(54) Device and method for parallel, automated cultivation of cells in technical conditions

(75) Inventors: Robert Puskeiler, Munich (DE); Dirk Weuster-Botz, Dachau (DE); Karl-Heinz Zacher, Buch am Buchrain (DE)

Correspondence Address: Andrew R Basile;Young & Basile, Suite 624, 3001 West Big Beaver Road, Troy, MI, 48084, US

(73) Assignee: Technische University Munchen, Munich (DE)

(21) Appl. No.: 10/540,299

(22) PCT Filed: Dec. 22, 2003
(86) PCT No.: PCT/EP03/14752

§ 371 Date: Dec. 8, 2005

(30) Foreign Application Priority Data
Dec. 23, 2002 (DE) 10260691.9

(51) Int. Cl. C12M 1/02

(52) U.S. Cl. 435/289.1; 435/304.2; 435/305.2

(57) ABSTRACT

The invention relates to a device for the cultivation of cells in liquid columns measured on a millimetre scale. Said device comprises a container for receiving a liquid culture suspension of the cells, a mixing element for mixing the culture suspension in the container around a mixing axis. The container and/or the mixing element are configured in such a manner that the speed of the flow is modified either locally and/or temporally along a streamline or flow line around the mixing axis.

The present invention relates to a device for cultivating cells, an arrangement of devices of this type, a agitation system suitable for this purpose and also a culture method for cells. Devices and methods of this type are required for cultivating cells on a millilitre scale. These are used in particular for parallel batches during strain or bioprocess development in the chemical industry, e.g. for reaction optimisation or catalyst optimisation, in the field of environmental protection, for the optimisation of sewage treatments or chemical or biological treatment of solid materials or exhaust air, or in the field of food technology.

Agitated flasks or mixing vessels have been used to date as reactors for cultivating cells in liquid columns on a millilitre scale, as is required in particular for parallel reactions for testing specific biotechnological techniques.

The standard parallel reactor in biotechnology is the agitated flask, with which simple batch experiments in a parallel batch have been implemented manually for the last century. Agitated flasks are mounted on agitated tables, set in a rotational movement with given eccentricity in incubators at a prescribed temperature at a specific agitation frequency. Due to the movement of the reaction vessel, mixing of the liquid which is contained in the reaction vessel and in which the biochemical reaction takes place, is effected.

Via surface aeration, the oxygen required for many biochemical reactions is fed into the liquid phase from the gas phase. High oxygen transfer rates are consequently only possible if a very large surface/volume ratio is set. This means that very large agitated flasks (1-21 volume) with very little reaction medium (10-20 ml) must be operated with the greatest possible eccentricity and agitation frequency (400 rpm). Under these conditions, oxygen transfer coefficients k_{L}a of up to 0.07 s⁻¹ are achieved.

The power input in an agitated flask is effected by the friction of the liquid on the inner wall of the rotating reaction vessel. Hence, a relatively uniform energy dissipation is effected.

The advantages of the agitated flask cultivation are simple handling and relatively low technical outlay.

Alternatively hereto, also stirred tank reactors can be used. Substantial reaction engineering differences between the reaction vessel, agitated flask, and the reaction vessel, stirred tank reactor,—the standard production reactor of biotechnology—are the lower oxygen transfer, the far smaller ratio of the maximum local energy dissipation to the average power input and the inadequate control of important process variables (such as for example pH or P_{O2}). This leads to the fact that reaction courses in most cases cannot be directly transferred from the reaction system, agitated flask, to the reaction system, stirred tank reactor, and hence, in bioprocess development, additional personnel and time-intensive sequential experiments in laboratory bioreactors are required.

Technical attempts to resolve this problem are the provision of parallel mixing vessel units with completely individual measuring and control technology. Parallel reactor systems with 4 or 6 stirred tank reactors with a volume of up to 0.5 l are commercially available. The obtained process data can generally be transferred readily to larger stirred tank reactors. The capital, personnel and time expenditure is however exceptionally high if a plurality of these parallel reactor units need to be used for bioprocess development.

A newer development is the operation of parallel small-scale reactors in an incubator with intermittent substrate dosage and parallel pH control (DE 197 09 603 A1). Either bubble columns or agitated columns with an operating volume of 200 ml are used as parallel small-scale reactors (DE 195 29 099 A1). Hence, oxygen transfer coefficients and volume-specific power input, as in the standard mixing vessel reactor, can be achieved. The number of parallel bioreactors is however restricted (≦16). A further parallelisation is practically impossible on the basis of this technology. For simultaneous concentration measurement of key components of the reaction medium (substrate or product concentration), parallel sampling and analysis systems are used (EP 0995 098 A1).

A further simple possibility of operating far more reactors in parallel is the use of microtitre plates in incubation agitators for batch cultivation of cells. Microtitre plates with 24, 48, 96 or more wells for cultivating cells have however, to an even greater extent, the same technical reaction restrictions as agitated flasks. In addition, the evaporation on this scale has proved to be problematic, since the relative evaporation volume flow relative to the initial volume, due to the very large surface/volume ratio and the small reaction volume (≦1.5 ml) is very much larger than for example in the agitated flask or stirred tank reactor.

In order to be able to rapidly implement new biological knowledge into technically achievable and economical methods, the sequential procedure to date with simple parallel batches in the agitated flask and subsequent optimisation of the reaction conditions in the controlled laboratory stirred tank reactor must be overcome. This is only possible if as large a number as possible of mixing vessel reactors can be parallel-operated in an automated fashion under technical, controlled reaction conditions.

In order to make parallelisation of mixing vessel reactors possible, these must be constructed as simply as possible and must be operated as far as possible without baffles. Ideal reaction vessels are for example sterilisable reagent glasses or microtitre plates with correspondingly large wells.

The power input can be effected simply by magnetic agitator drives on this scale. In technical agitation reactors, the oxygen transfer into the reaction medium with volume aeration is determined primarily by the power input of the impeller and secondarily by the superficial gas velocity. Primary dispersion of the gas phase, as is effected in the standard stirred tank reactor via a gas distributor on the reactor base, can however only be achieved in a very complex manner in millilitre parallel-operated stirred tank reactors. The reaction vessels would have to be provided with an individual gas supply and a gas sparger. The gas sparger would have to ensure that the desired superficial gas velocity was achieved exactly in each of the parallel reaction vessels.

It has only been possible to date to implement either a large number of simple, uncontrolled parallel reactions under non-technical conditions in microtitre plates or in agitated flasks or to operate a relatively small number of bioreactors under controlled, technical conditions.

Since it is necessary to achieve a high gas transfer into the reaction vessels, one option is to implement the sterile gas supply into the vessels from above. For this purpose, the simplest sterile boundary would be use of a sterile filter as a cover of the individual reactors or of an entire arrangement of reactors. Such a sterile filter, in addition to the mechanical barrier for contaminants, would however require to have good gas transfer properties in order to avoid oxygen limitation in the reaction vessels.

Furthermore, the cover of the reaction vessel represents the only possibility for intervening in the reaction course, for example for adding substrate, titration media or inductors during the reaction, for sample removal for process control or for introducing measurement probes. In order to be able to implement these interventions perfectly in a technically sterile manner, the sterile filter is usually configured as a septum. The gas permeability of septums, which are generally based on silicon, is however inadequate so that both functions cannot be fulfilled by one material. It is therefore problematic to have a simple and guaranteed sterile access to the reaction vessel or vessels, this access however being intended to be sterile in all circumstances.

It is therefore the object of the present invention to produce a device for cultivating cells in liquid columns on a millilitre scale, with which, on the one hand, a high gas and power input is achieved and, on the other hand, individual or also a large number of devices of this type can be operated effectively in a parallel manner. Furthermore, it is the object of the present invention to make available an arrangement of this type of parallel devices and also an agitation system with which the desired high power input and gas transfer can be achieved. The object of the present invention is in addition to make available corresponding culture methods of cells in liquid columns on a millilitre scale.

This object is achieved by the device according to claim 1, the agitation system according to claim 21, the arrangement according to claim 31 and also the method according to claim 49. Advantageous developments of the respective device, arrangement, agitation system or of the method according to the invention are given in the respective dependent claims.

With the solution according to the invention, it is possible to operate individual or also a multiplicity of automated stirred tank reactors, for example 24, 48 or 96 or more stirred tank reactors, both for strain and also for bioprocess development in a time-effective manner under technical reaction conditions. Hence, the parallel, automated cultivation of cells on a millilitre scale under individually controlled reaction conditions, such as temperature, hydrogen ion activity, oxygen feed, power input and also media supply is possible, so that reaction courses, as are achieved in the standard stirred tank bioreactor, can be implemented in a parallel batch. There is thereby understood by millilitre scale advantageously a range for the mixed liquid volume of 0.5 to 50 ml, preferably from 1 to 30 ml, preferably from 5 to 20 ml. By means of the device according to the invention and the method according to the invention, direct transfer of the thus obtained (fed batch) process courses from the millilitre scale according to the invention to the litre scale (and vice versa) is possible. The millilitre agitation reactors according to the invention permit an equally efficient oxygen supply of organisms in liquid culture as stirred tank reactors of a larger scale with volume gassing.

When using the stirred tank reactors according to the invention, parallelisation of a large number of bioreactors in one bioreactor block is possible, operation thereof being able to be automated for the first time by using laboratory robots, for example pipetting robots and the like and thus an efficient individually controlled parallel operation is made possible. Only the device according to the invention and also the cover according to the invention make possible the use of a laboratory robot and hence a quantum leap in obtaining relevant process data.

If the laboratory robots are operated with suitable screening and optimising routines, then process optimisation, for example with respect to media composition, induction methods and dosage profiles, can be achieved systematically and with high time efficiency. The complete digitalisation of the parallel process development makes possible furthermore a novel data transparency and data availability.

Since the volumes of the mixing vessel reactors, with which nevertheless meaningful information about the respective process course can be obtained, can now be greatly minimised, for example instead of 500 ml now merely 5 ml, with the same total volume by using 100× more reaction vessels, a multiple of information can be obtained or with the same quantity of information the time expenditure can be minimised by the achieved automation. When using suitable experimental planning algorithms, a quantum leap in the effectiveness of the bioprocess development can be made possible.

The present invention is based crucially on the fact that it was detected that the gas transfer from the surface of the liquid column into the liquid column in a mixing vessel reactor is improved as a result of the fact that either the container and/or the agitation system are configured in such a manner that the flow velocity is modified locally and/or temporally along a streamline or flow line which, in the case of a stirred tank reactor, extends in a circle. This leads to a spatially and/or temporally pulsating Bernoulli effect. This can lead for example to a flow field of the culture suspension which is directed towards the base of the mixing vessel reactor (container), which leads to intensive entry of gas bubbles. There are described as streamlines thereby lines in the flow, the direction of which is identical to the direction of the velocity sector at each ramming point. There are described as flow line lines through which liquid particles flow.

On the basis of this knowledge, it is now possible to configure either the container (stirred tank reactor) and/or the agitation system in a suitable manner.

It is possible, on the one hand, to configure the container itself such that its inner volume has no rotationally symmetrical shape. Where the liquid flow then widens, there is a region of low flow velocity and hence high pressure, whilst where the liquid flows through between the agitation system and the container wall, a region of high flow velocity and hence low pressure is present there. Hence, a flow velocity which varies spatially along the circumference of the container or a liquid pressure which varies is given.

The same effect is achieved if the agitation system is disposed off-centre or eccentrically within the container which has any shape or is shaped rotationally symmetrically. In this case, spacings between the agitation system and the wall of the container are again produced, which spacings vary along the circumference of the container and consequently induce different flow velocities and pressure ratios.

A further possibility for achieving this pulsating Bernoulli effect is to dispose baffles along the circumference of the container. There are thereby understood by baffles elements which are situated in the flow of the mixed liquid and thus represent a flow resistance for this. Baffles lead to a narrowing of the flow cross-section. Since the spacing between impeller and wall of the vessel is greater than between impeller and baffle, again regions of high flow velocity are formed between impeller and baffle and regions of low flow velocity between impeller and free wall regions. The baffle must thereby be disposed not in the rotational plane of the agitation system but can be disposed below, in the rotational plane or also or in addition also above the rotational plane of the agitation system. In all these cases a pulsating Bernoulli effect is produced. The baffles can advantageously be configured in one piece with the mounting of the agitation system or in one piece with the container, for example in the injection moulding method.

The gap spacings should thereby be chosen such that an adequate pulsating Bernoulli effect is produced, but the shear forces should not become so great that the cells contained in the suspension are destroyed. Gap spacings >0.05 mm, preferably >0.1 mm and/or <20 mm, preferably <3 mm, are particularly suitable for this purpose. There are suitable as containers standard mixing flasks, reagent glasses or also the wells of a microtitre plate or a specially prepared plate with the same cavity arrangement with adequate diameters.

A further possibility of producing a pulsating Bernoulli effect resides in configuring the agitation system in a suitable manner. For this purpose, a boring is introduced into the agitation system which boring extends from the underside and/or side wall of the agitation system to a side wall or to the upper side of the agitation system. Advantageously the boring extends at an angle α with 0°≦α<90° relative to the rotational or central axis of the agitation system, this angle opening upwards.

Advantageously also further through-channels with a corresponding opening can extend downwards from the upper side and/or side wall of the agitation system to the side wall or underside thereof.

These channels can also extend only partially through the agitation system at an angle α and then meet a further channel which, with respect to the plane perpendicular to the rotational axis, extends in this plane or linked at an angle <90° upwards or downwards to this plane and discharge in said further channel which for its part ends at the lateral outer wall of the agitation system with an opening.

Through borings of this type, a flow is likewise induced from the base of the container to the side wall of the agitation system, which leads to an altered pressure at the opening of the borings or channels which is orientated away from the rotational axis and thus induces a pulsating Bernoulli effect.

Advantageously, the vessels have a closure or cover which covers the vessel or an arrangement of vessels in a sterile manner. On the one hand, gas distributor structures can be introduced into these closures for the supply of sterile gas. For this purpose, the closure can comprise for example a double base plate, the gas distributor structure being disposed in the intermediate space between the two plates of the double base plate. The closure can also comprise one or more plates, the gas distributor structures being disposed on the underside of t he lowermost plate.

Advantageously, the gas distributor structures are set up in such a manner that, starting from a central gas supply, individual channels lead to the respective containers as branches. Advantageously the channels are thereby guided such that they both have the same cross-sections, the same length and the same number of bends or kinks. Consequently, a uniform gas pressure is effected on all the containers. Merely the branches can either be set up thereby in the same manner or else the entire system can be.

Furthermore, the closure advantageously has an opening for each individual container, through which sterile gas supplied externally of the container flows. This opening can for example be a tube through which a cannula or any other elongated sample removal or sensor unit can be introduced. Since this is then effected in counter-flow, it is merely required to sterilise the respectively introduced unit in advance and then to introduce the tube into the suspension in order to avoid contamination of the reaction vessel.

Furthermore, the closure can have webs which, in an arrangement of containers, isolate the individual containers from each other in a sterile manner. A further web, which is likewise assigned to the respective container, can be configured such that it is immersed into the suspension and thereby separates the inlet for the sterile gas from the above-mentioned outlet. This leads to the fact that sterile gas is forced into a route through the culture suspension and consequently gassing of the culture suspension is further improved.

Examples of the device, arrangement of device, agitation systems and associated methods according to the invention are described subsequently.

There are shown

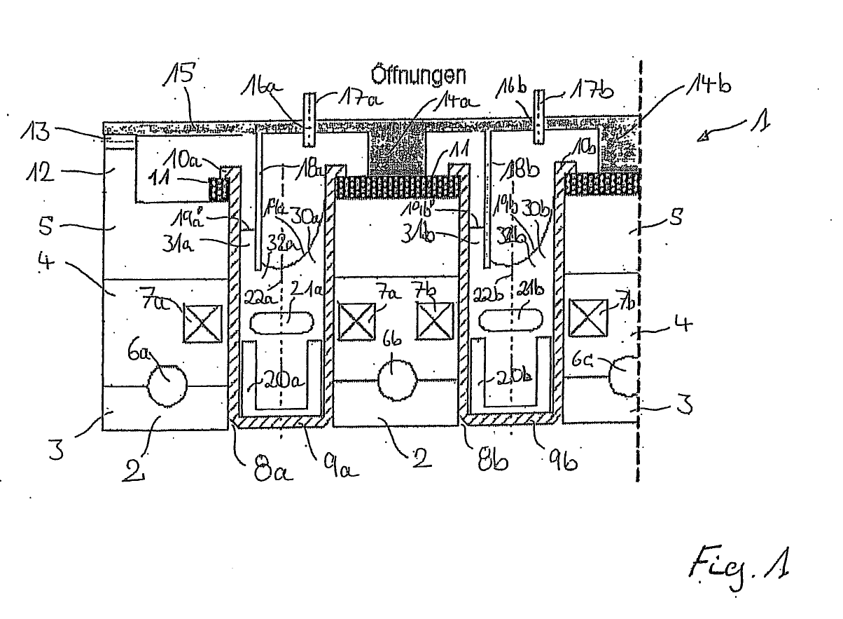

FIG. 1 a longitudinal section through an arrangement (bioreactor block) of bioreactors;

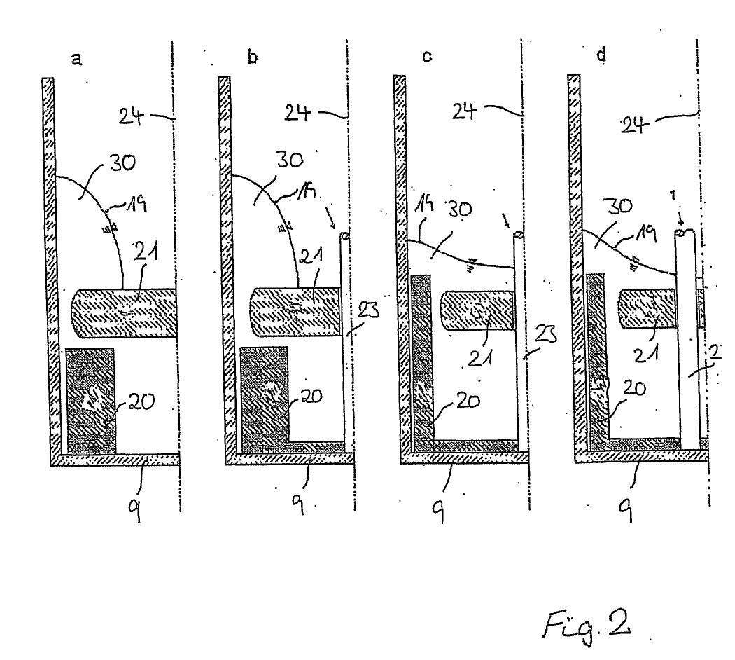

FIG. 2 various variants of the arrangement of baffles and agitation systems in a bioreactor;

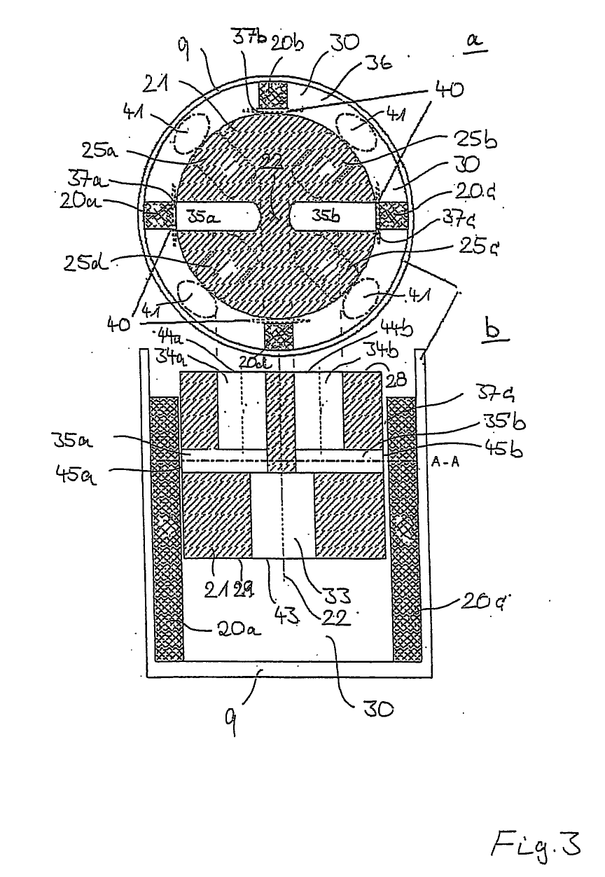

FIG. 3 the arrangement of a agitation system according to the invention and baffles in a bioreactor;

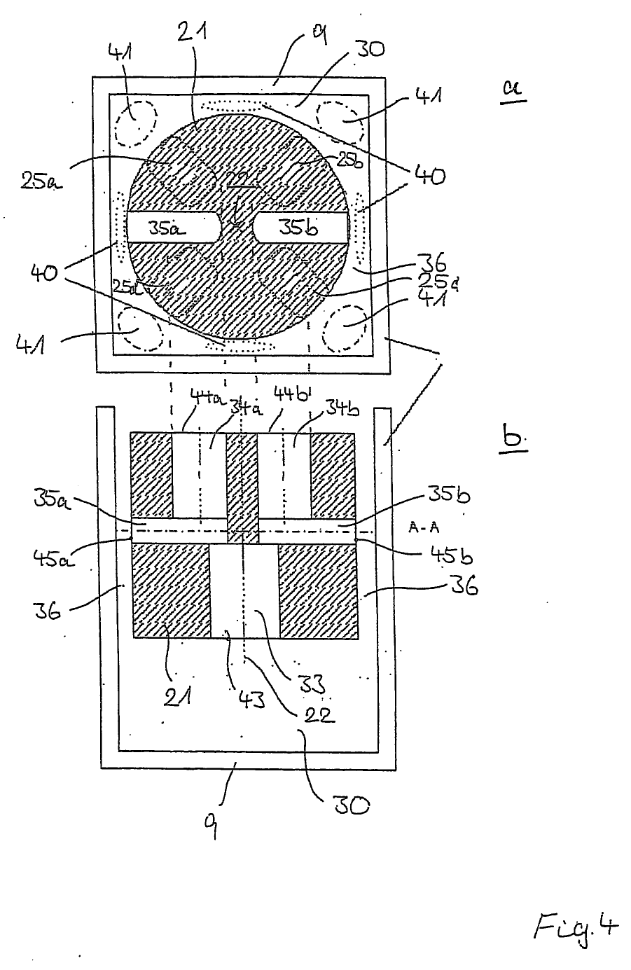

FIGS. 4 to 6 further devices according to the invention;

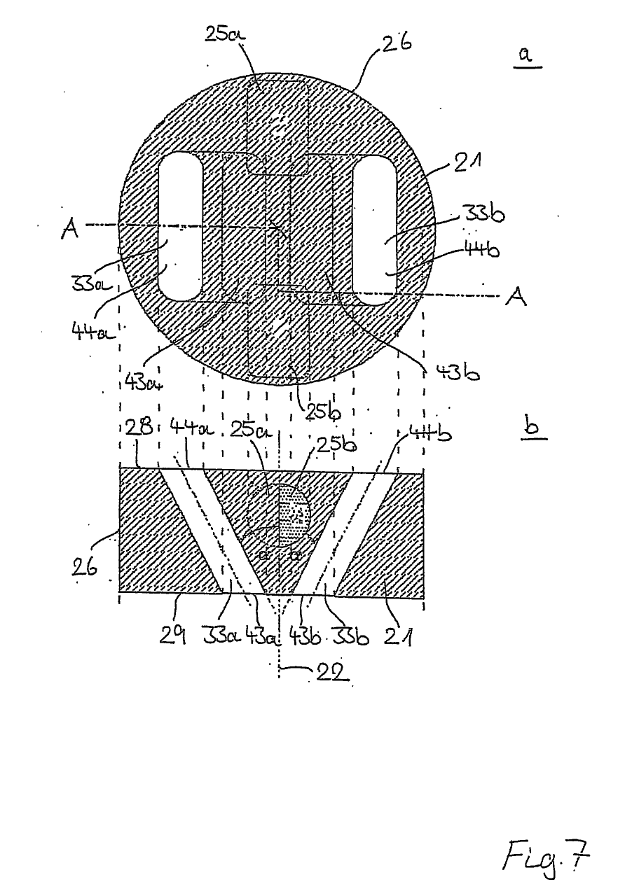

FIG. 7 an agitation system according to the invention;

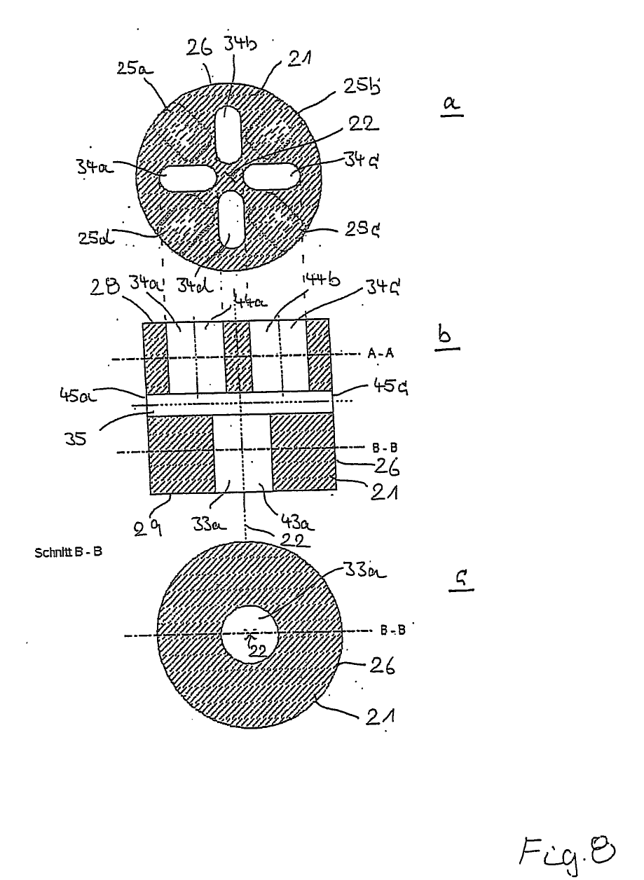

FIGS. 8 to 13 further agitation systems according to the invention;

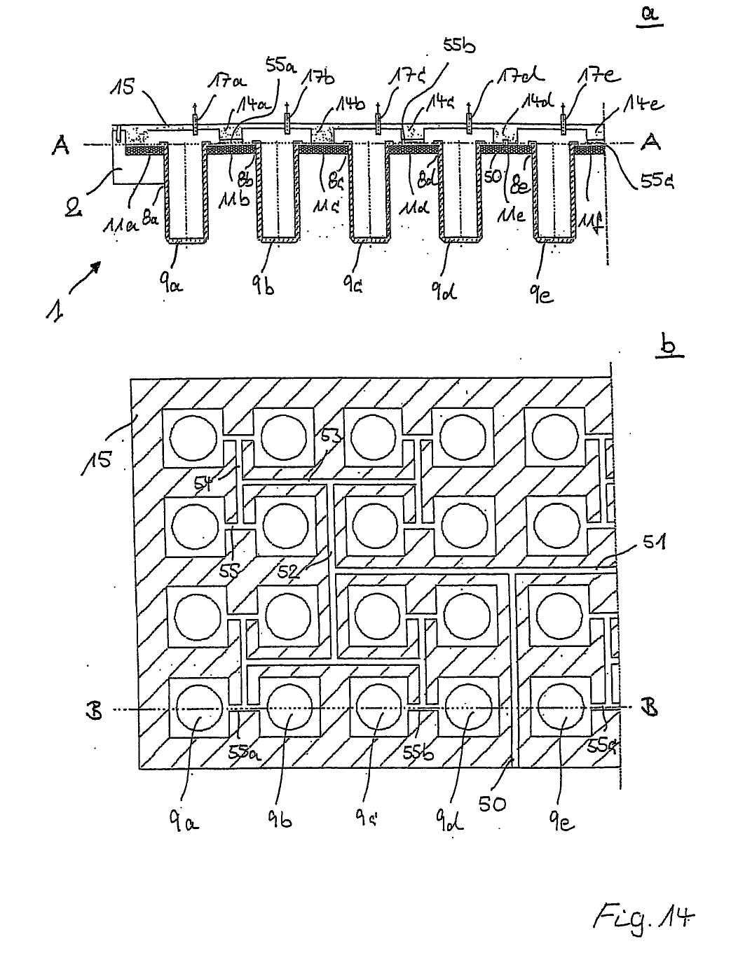

FIG. 14 a further arrangement of bioreactors and the structure of the associated closure;

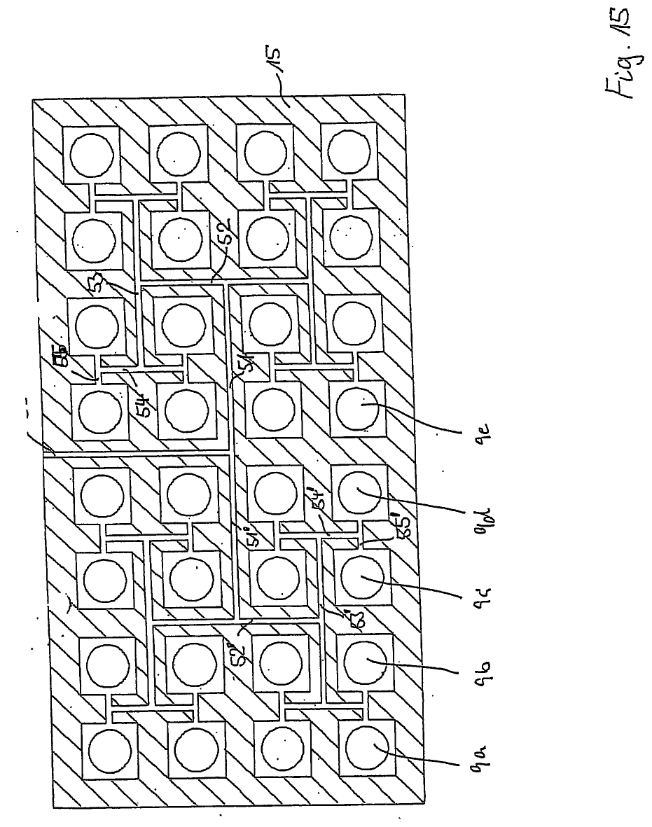

FIG. 15 the structure of a further closure;

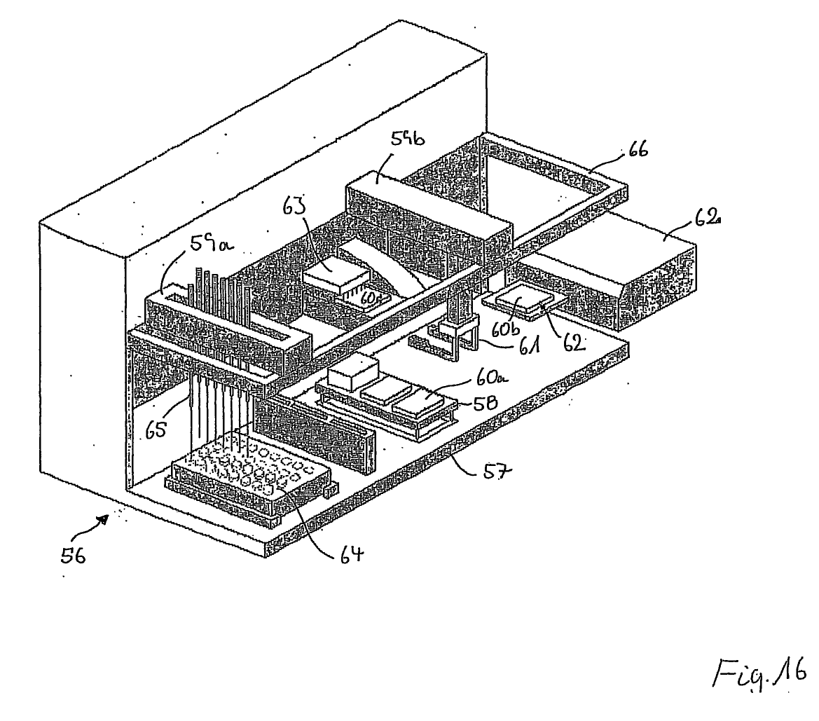

FIG. 16 a general arrangement with pipetting robot;

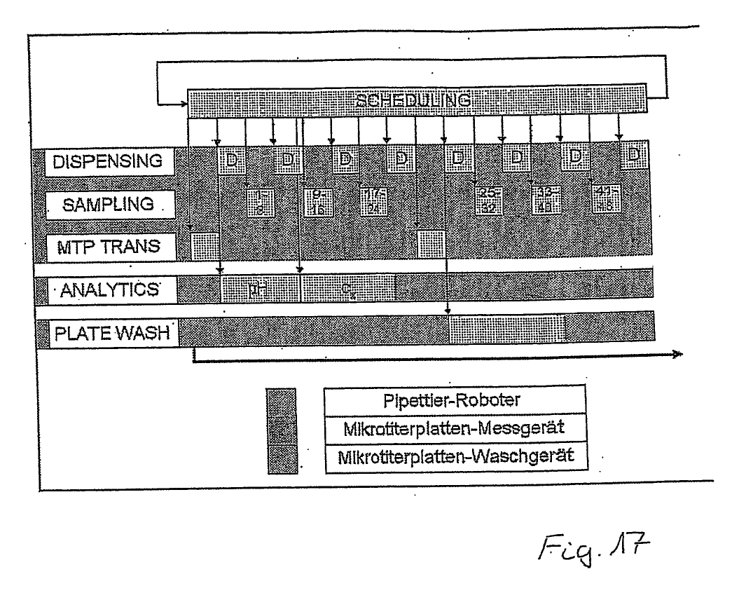

FIG. 17 the principle course of a parallel control of a bioreactor arrangement;

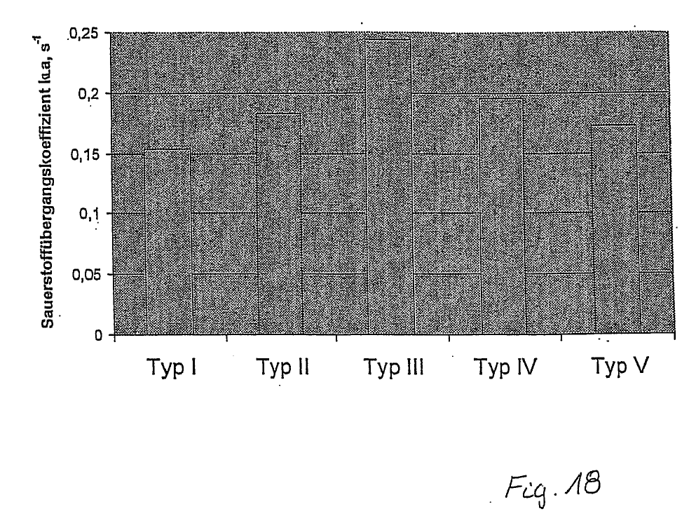

FIG. 18 maximum oxygen transfer coefficients for magnetic agitation systems according to the invention of varying types;

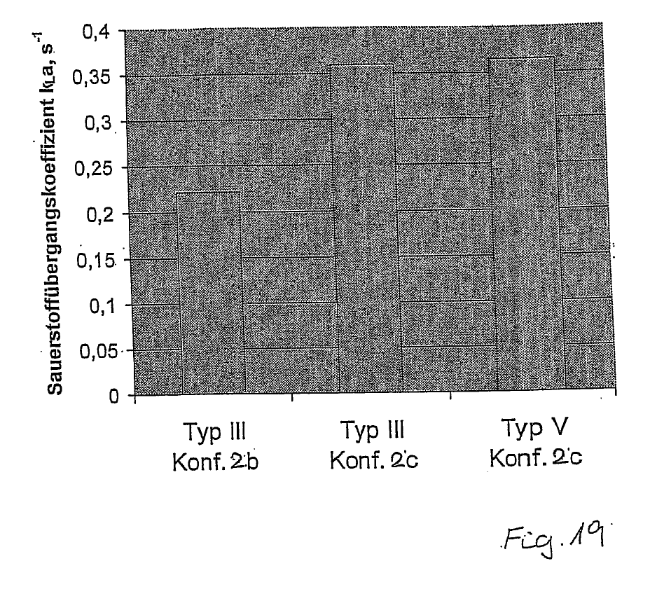

FIG. 19 oxygen transfer coefficients for magnetic agitation systems of different types according to the invention;

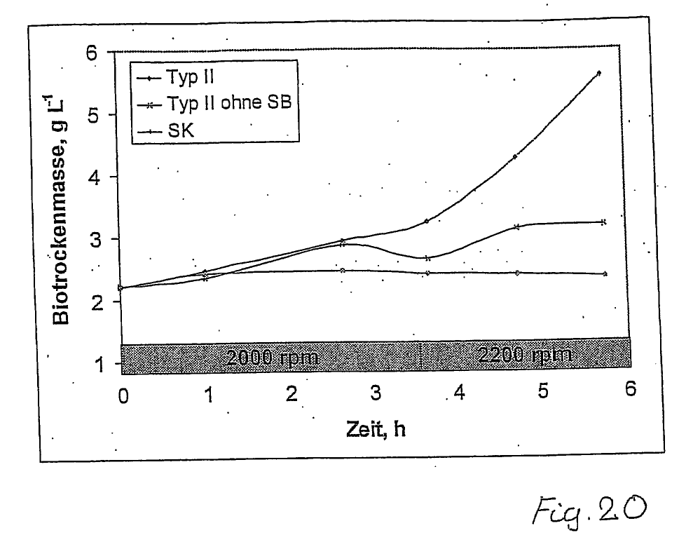

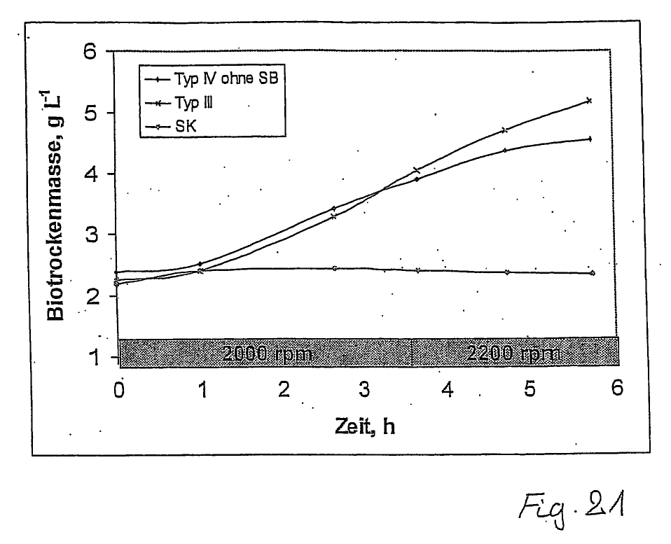

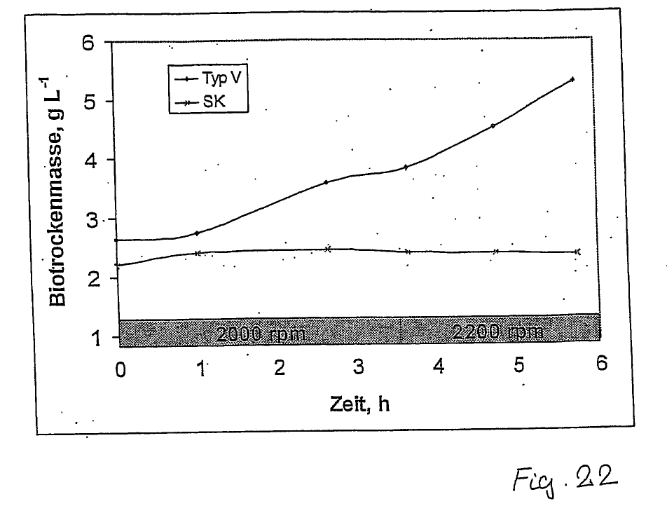

FIGS. 20 to 22 the results of the cultivation of Escherichia coli in agitation systems according to the invention of a different type;

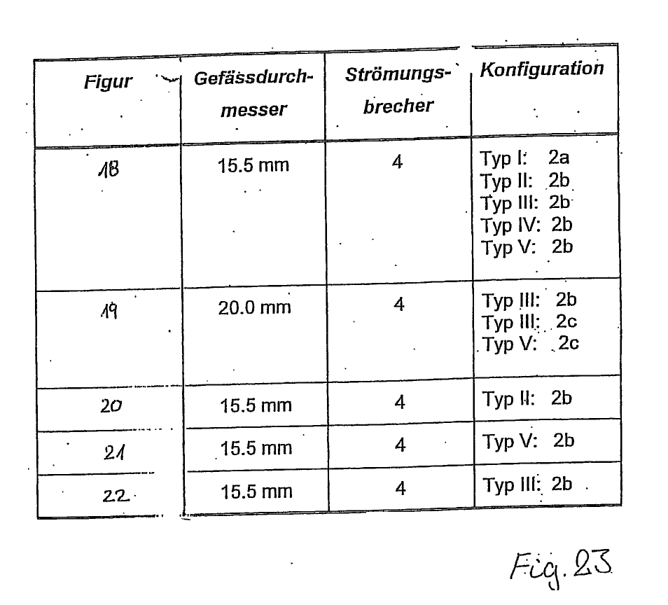

FIG. 23 a Table of the agitation systems used for the measurements according to FIGS. 18 to 22;

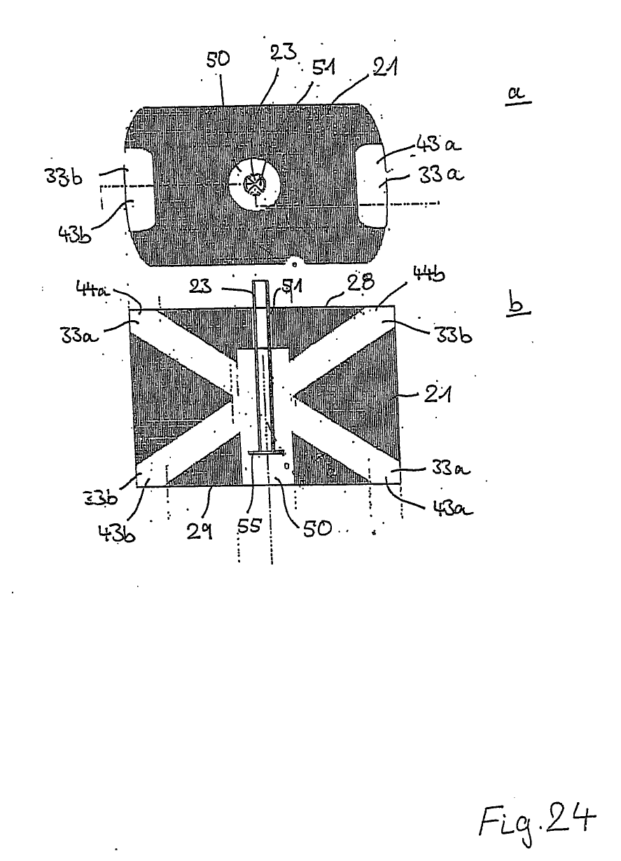

FIGS. 24 to 26 further agitation systems according to the invention;

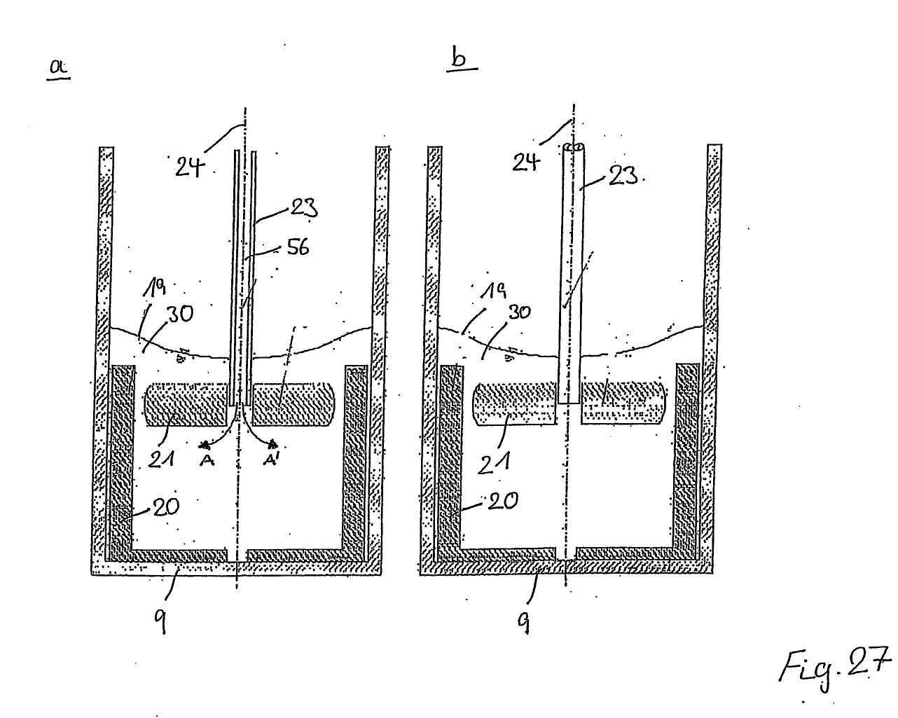

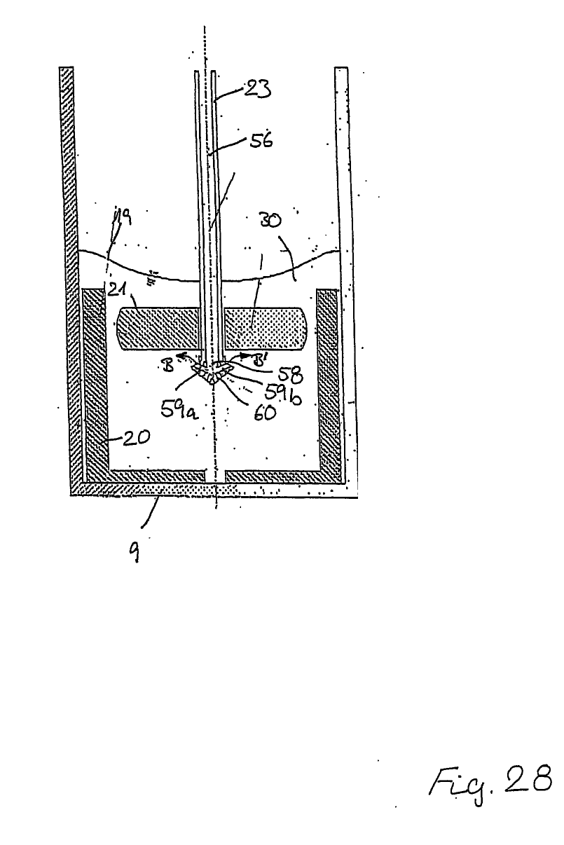

FIGS. 27 to 28 further devices according to the invention;

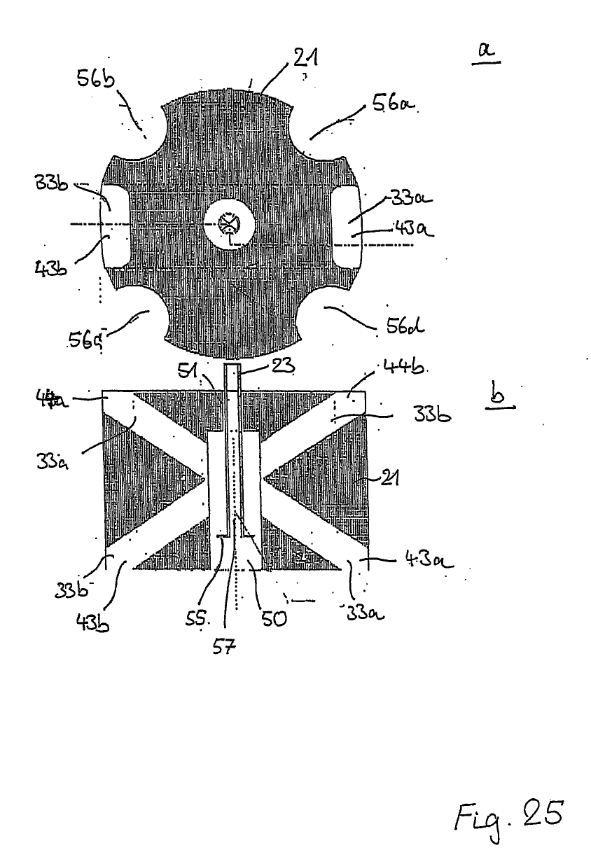

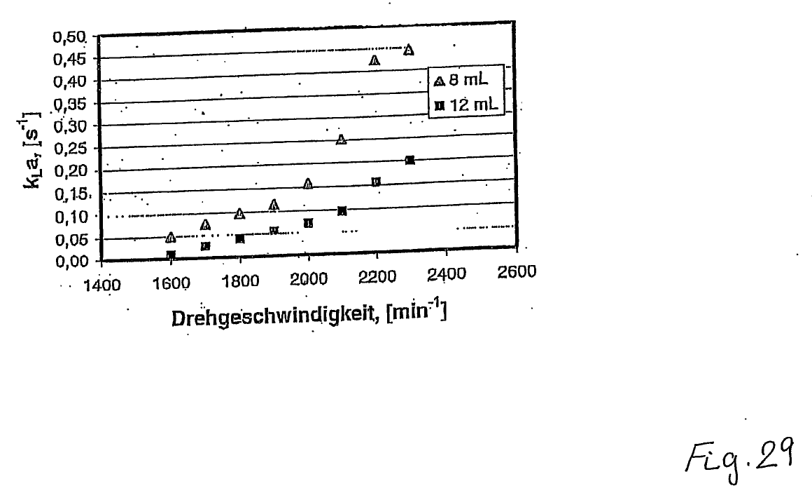

FIG. 29 maximum oxygen transfer coefficients for an agitation system according to FIG. 25;

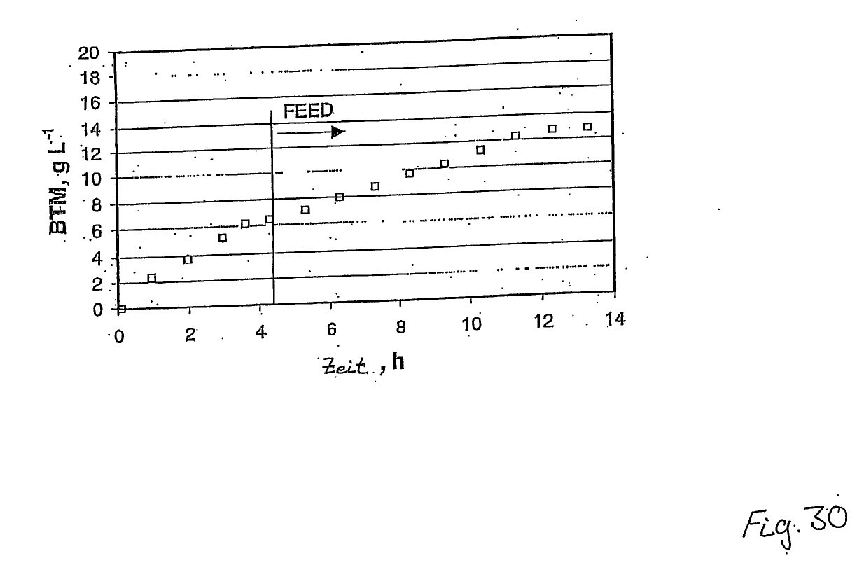

FIG. 30 biomass dry concentrations achieved with an agitation system according to FIG. 25.

In the following, the same or similar reference numbers are used in all Figures for the same or similar elements.

FIG. 1 shows an arrangement according to the invention of reaction vessels 9a, 9b as containers in a module system 2, which is described in the following in total as bioreactor block 1.

This bioreactor block 1 contains up to 96 cavities or borings 8a, 8b, these being able to be disposed in different formats, for example 4×3, 4×5, 8×3, 8×6 or 8×12 borings. The diameters of the borings 8a, 8b are advantageously between 10 and 35 mm. They are disposed in the bioreactor block 1 such that correspondingly dimensioned micro-reaction vessels 9a, 9b can be introduced into the latter in a form-fit. The bioreactor block 1 is thereby constructed from a multiplicity of horizontal layers 3, 4, 5, the lowermost layer 3 forming a baseplate, the layer 4 thereabove a central part and the layer 5 thereabove an upper part. Borings 6a, 6b, 6c are disposed between the baseplate 3 and the central part 4 through which, as heat exchanger, a fluid at a suitable temperature flows and which thus moderates the temperature of the entire bioreactor block 1. Furthermore, the borings 8a, 8b surrounding magnetically inductive magnetic drives 7a, 7b are disposed in the central part 4, as are known for example from U.S. Pat. No. 4,568,195. The upper part 5 contains a laterally projecting edge 12 along the outside of the entire bioreactor block 1, into which a sterile gas supply 13 is introduced for supplying sterile gas from the outside into the bioreactor block 1.

In the inner region of the bioreactor block 1, a distance disc 11 can be placed on the upper part 5. Since the individual mixing vessel 9a, 9b has an annular flange 10a, 10b on its upper side or upper edge, this flange 10a, 10b is supported on the distance disc 11. By choice of a suitably thick distance disc 11, the height of the reactor vessel 9a, 9b can be adjusted. Hence, the mixing height of a magnetic mixer 21a, 21b disposed in the vessel 9a, 9b above the base of the reaction vessel 9a, 9b is then defined. A one-piece baffle 20a, 20b is disposed in the respective reaction vessel 9a, 9b lying on its base, said baffle narrowing the cross-section of the reactor vessel at two positions on the circumferential line of the reactor vessel 9a, 9b. The baffle 20a, 20b ends with its upper edge in the present example below the mixing plane of the agitation system 21a, 21b. In other examples, the baffle can also however extend laterally beside the agitation system or even protrude upwardly beyond the latter. It is also possible that the baffles are disposed only above and/or in the mixing plane of the agitation system.

The lid or cover 15 applied on the reactor block 1 has central webs 14a, 14b which extend up to the distance disc 11 and thus isolate the individual reaction vessels 9a, 9b from each other in a sterile manner as separating walls. Furthermore, webs 18a, 18b are provided which extend into the reaction chamber 9a, 9b and separate this likewise as separating walls into two compartments. Finally, the cover 15 also has another boring 16a, 16b respectively, through which respectively one tube 17a, 17b extends. This tube 17a, 17b represents a constantly open connection between the outside of the bioreactor block 1 and respectively one of the reactor vessels 9a, 9b.

If now a culture suspension 30a, 30b is introduced into the respective vessels 9a, 9b, then the web 18a, 18b separates the surface 19a, 19b of the liquid 30a, 30b into two regions 19a, 19a′ or 19b, 19b′ which are separated from each other. If now the mixer 21a or 21b is set in rotation, then a convex liquid surface 19a or 19b is formed because of co-rotation of the liquid. This effect is not so pronounced for the liquid 30a or 30b at the surface 19a′ or 19b′ and is not represented here further.

If now a gas under excess pressure is guided via the sterile gas supply 13 to the surface 19a′ then, in order to again leave the container 9a, said gas must flow through the left member 31a of the liquid column below the web 18a into the right member 32a of the liquid column and from there again leave the vessel via the tube 17a. Since the flow though the tube 17a is constantly in this way from inside to outside, the reaction vessel 9a is sterile although the tube 17a is open and forms a constantly open access to the vessel 9a. The same applies correspondingly for the reaction vessel 9b.

Advantageously, interventions into the reaction course can now be implemented readily via the tube 17a or 17b. This means for example that substrate or titration media or inductors can be added via the tube 17a, 17b, that samples can be removed or measurement probes, for example pH electrodes, can be introduced into the liquid 30a or 30b. The introduction of corresponding probes is thereby effected in counter-flow to the outflowing sterile gas, so that contamination of the vessel 9a or 9b is avoided.

The cover 15 therefore produces a sterile cover for the bioreactor 1 with a central gas feed 13 via a sterile filter. An individual distribution of the sterile gas via gas distributor structures leading to the individual millilitre stirred tank reactors can likewise be effected.

The convective air flow through the tube 17a, 17b therefore prevents, in operation, the introduction of extraneous germs via the surrounding air. The open conducting pipe 17a, 17b is manufactured here for example from aluminium and is consequently also suitable as access with sterile pipette tips or piercing cannulae.

If gas distributor structures are inserted in the sterile cover, then these should be configured such that cross-contamination by aerosol entrainment or foam formation is precluded.

The bioreactor block 1 and the cover 15 are configured in such a precisely adapted manner that they can be assembled inside a sterile workbench, if necessary after sterile filling of the individual reactors 9a, 9b with reaction medium 30a, 30b, to form a functional unit.

The sterilisation of the bioreactor block 1 and of the cover 15 can either be effected together in an autoclave or also as individual components. In order to sterilise the bioreactor block 1 in the autoclave, a cost-intensive encapsulation of the inductive drives 7a, 7b is necessary in order to make direct autoclaving possible.

Alternatively, as also represented here, bioreactor inserts 9a, 9b can be used (corresponding to microtitre plates) which can be sterilised separately from the bioreactor block 1. These bioreactor inserts 9a, 9b can also be configured as sterile single-use or disposable articles as long as the material and production costs thereof are low.

FIG. 2 shows, in the partial pictures a to d, the use of baffles 20 and agitation systems 21 for generating a pulsating Bernoulli effect. Only the left half of a reaction vessel 9 is thereby illustrated respectively.

In FIGS. 20a and 20b, the upper edge of the baffle 20 is below the mixing plane of the agitation system 21.

In FIG. 2a, the agitation system is mounted centrally in the vessel 9, the mounting being effected magnetically inductively.

In FIG. 2b, the magnetic agitation system 21 is mounted via a shaft 23, via which it can also be actuated.

In FIGS. 2c and 2d, the baffle 20 extends laterally beyond the rotational plane of the magnetic agitation system 21.

In FIG. 2c, the agitation system 21, just as in FIG. 2b, is mounted via a shaft 23 and is if necessary also actuated via this or magnetically. In FIG. 2d, the shaft 23 is mounted within the mixing vessel 9 eccentrically outwith the central axis 24 of the vessel 9 so that the pulsating Bernoulli effect generated by the baffle is increased even further here.

In the individual cases of FIGS. 2a and 2d, a differently formed liquid surface 19 of the liquid 30 is hence formed.

In all the examples 2b to 2d, it is possible, as long as the shaft 23 does not serve for actuation of the magnetic agitation system 21, to manufacture the shaft 23 and the baffle 20 in one piece and to insert it as a unit into the reaction vessel 9 in a precise fit.

Examples of agitation systems according to the invention and reaction vessels according to the invention are now illustrated in the following. By using a suitable agitation system, the possibility exists in principle of dispensing with a primary dispersion of the gas phase via a gas distributor, as in the state of the art, since small reaction vessels, in comparison to laboratory stirred tank reactors, have a far higher surface/volume ratio.

In the present invention, suitable advantageously steam-sterilisable magnetic agitation systems have been developed which effect axial conveyance from the liquid surface to the base of the reaction vessel (absorption of the gas phase) and effective dispersion of the gas phase into as small as possible gas bubbles with a high oxygen transfer area (high local energy dissipation) in the reaction medium and also release of the spent gas bubbles at the liquid surface. These magnetic agitation systems have a basic body which can be manufactured advantageously from Teflon and contain one to four magnetic cores (ferrite or rare earth magnets, such as e.g. SmCo (samarium cobalt) or NdFeB (neodymium iron boron)) as actuation means. The subsequently represented magnetic agitation systems advantageously have the following dimensions and shapes:
circular cylindrical magnetic agitation systems 3 to 20 mm diameter, height from 2 to 25 mm.
egg-shaped magnetic agitation systems of a round central cross-section (diameter 15 mm), length from 3 to 20 mm.
cuboid magnetic agitation systems 3×8 mm to 9×20 mm surface area and heights of 4 to 25 mm.

These basic bodies are advantageously provided with borings or channels. These are between 3 and 20 mm long and have diameters which should be adapted to the size of the agitation system, advantageously from 0.5 to 5 mm, advantageously from 0.5 to 3 mm. Different arrangements of the borings can be hereby produced:
channels extending at an upwardly open angle α to the vertical induce an annular flow in the reaction vessel.
channels extending at an upwardly open angle a to the vertical, before their exit into the liquid, meet air channels extending at an angle β to the horizontal.
vertically extending channels with a spacing of 0-3 mm encourage degassing of the liquid phase.

These magnetic agitation systems are thereby accelerated to speeds up to 4000 rpm, for example by a suitable magnetic rotary field or by a shaft.

Absorption of the gas phase into the reaction vessels with these agitation systems begins at a minimum rotational speed of the magnetic agitation system and becomes stronger by increasing the rotational speed. This minimum rotational speed is dependent upon the magnetic agitation system which is used, upon the position of the magnetic agitation system below the stationary liquid surface and upon the material properties of the liquid.

A particularly effective absorption of the gas phase and dispersion in gas bubbles in the reaction medium can be effected in reaction vessels with baffles which are disposed along the vessel wall in the circulating liquid flow. These advantageously one to four baffles can be disposed either below and/or above or over the entire vessel height on the vessel wall.

The magnetic agitation system is preferably operated in a self-centring manner, in a suitable rotating magnetic field. However, as shown in FIG. 2, mounting of the magnetic agitation system can also be effected on a shaft which is fixed in the reaction vessel.

FIGS. 3 to 13 show different embodiments of mixing vessels or agitation systems according to the invention.

FIG. 3 shows in FIG. 3b a mixing vessel 9 in which, on respectively oppositely situated sides, in total four baffles 20a to 20b are disposed. These are distributed at a 90° spacing on the circumference of the mixing vessel 9 and extend into the rotational plane of the agitation system 21 disposed in the vessel 9. The agitation system 21 rotates about its central axis 22 and is disposed centrally in the vessel 9. Starting from its underside 29, it has a boring 33 with a lower opening 43 which extends upwardly in a perpendicular direction, i.e. at an angle of 0° to the rotational axis 22. From the upper side 28 of the rotational element 21, two borings 34a and 34b with openings 44a and 44b on the upper side 28 of the rotational element 21 extend downwardly. All the borings 33, 34a and 34b discharge into horizontal borings 35a and 35b which, offset relative to one another by 180° extend up to the lateral external side of the agitation system 21 and form openings 45a and 45b there.

If the agitation system 21 rotates about its central axis 22 in a liquid 30, the channel 33 induces an annular flow in the reaction vessel together with the horizontally extending channel 35a. The channels 34a and 34b for their part absorb gas from above and likewise lead to improved gassing of the liquid 30 situated in the vessel 9.

FIG. 3a shows a cross-section along the line A-A in FIG. 3b through the entire arrangement. On the assumption that the agitation system 21 rotates about the axis 22 in the clockwise direction (this assumption is also maintained in the following Figures), there are now formed between the baffles 20a to 20d and the agitation system 21, respectively narrow gaps 37a to 37d with regions 40 with a high flow velocity and hence low, liquid pressure. Between the baffles 20a to 20d along the circumference of the vessel, a large gap 36 is present between the agitation system 21 and the wall of the vessel 9, in which accordingly regions 41 with low flow velocity and hence high hydrostatic pressure occur. In total, the operation of such a reaction vessel 9 leads to a local variation in the flow velocity i.e. to a locally pulsating Bernoulli effect.

Via the borings 33, 34a, 34b, 35a and 35b, temporally pulsating variations in the flow velocity which run along the openings 45a and 45b in the vessel are furthermore induced and lead likewise to the aeration of the reaction volume.

The actuation of the agitation system 21 is effected magnetically inductively via magnets 25a to 25d which are incorporated in the agitation system 21.

FIG. 4 shows an arrangement with a agitation system 21 which is identical to FIG. 3, here however, as can be detected in FIG. 4a, with the section along the line A-A in FIG. 4b, the reaction vessel having a rectangular cross-section. Baffles are not present. In this case, the gap width between the agitation system which is constructed rotationally symmetrically and the wall of the mixing vessel 9 changes spatially periodically, since the gaps in the corners of the mixing vessel 9 are broader than between the centre of the respective wall of the mixing vessel 9 and the agitation system 21. This leads in turn to regions 40 with a high flow velocity in the centre of the walls and regions 41 with a low flow velocity in the corners of the vessel 9. The effect of this thus spatially pulsating Bernoulli effect is increased by using a agitation system 21 with the prescribed borings which induce a temporally pulsating Bernoulli effect.

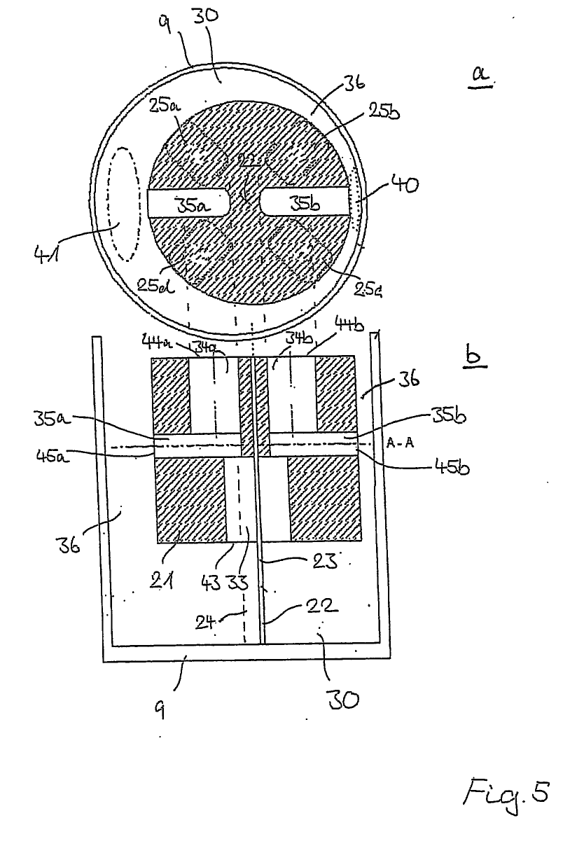

FIG. 5 shows in turn the same agitation system 21 which is now disposed however in a rotationally symmetrical mixing vessel 9. However, the arrangement of the agitation system 21 is effected eccentrically to the central axis 24 of the vessel 9 on a shaft 23. It is now ensured by the eccentric arrangement that the gap spacing 36 between the wall of the mixing vessel 9 and the agitation system 21 is greater on one side of the vessel than on the other side of the vessel. Correspondingly, zones 41 of low flow velocity 41 are again formed in the region of the large gap and zones 40 of high flow velocity in the region of the narrow gap. Here also, the Bernoulli effect, pulsating with a period of 3600, is increased by the borings 33, 34a, 34b, 35a, 35b, . . . in the agitation system 21.

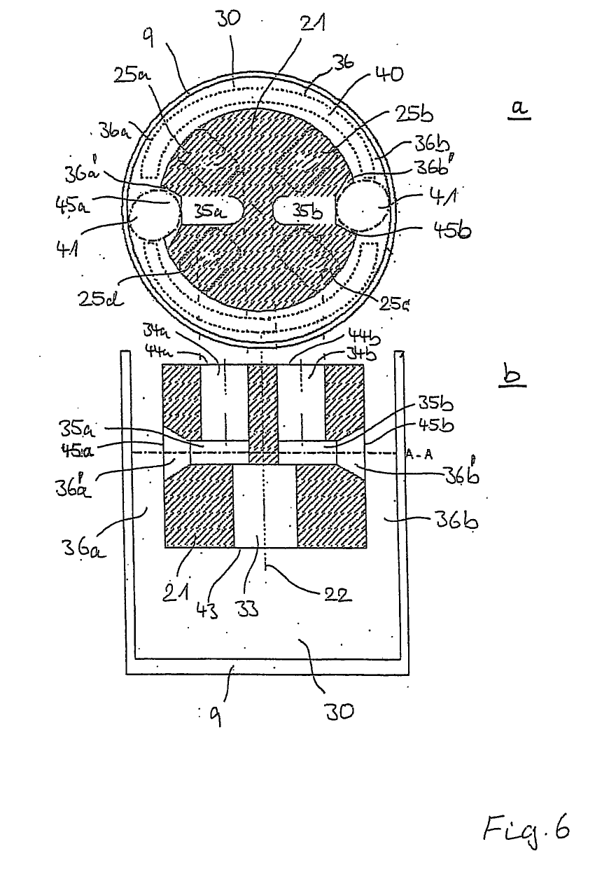

In FIG. 6 also, a similar agitation system 21 is used as in the previous Figure. In this case, a cylindrical reaction vessel 9 is present again and a cylindrical agitation system 21. The spacing 36 between the wall of the mixing vessel 9 and the agitation system 21 is now almost identical on the full circumference, with two exceptions. For the horizontal borings 35a and 35b are conically widened in the present agitation system 21 in the region of their lateral openings 45a and 45b. In these regions respectively, a widening 36a′ or 36b′ of the gap 36 is present so that zones 41 of low flow velocity are formed there. Therefore, these zones 41 of low flow velocity now circulate with the agitation system 21 within the vessel 9. This leads to a locally and temporally pulsating flow field and hence in turn to the desired pulsating Bernoulli effect.

FIG. 7 shows the simplest form of a agitation system 21 with diagonally outwardly extending borings 33a and 33b. The borings 33a, 33b begin at the underside 29 of the agitation system 21 with an opening 43a, 43b and end at the upper side 28 of the mixer 21 in an opening 44a or 44b. FIG. 7b thereby shows a section along the line A-A from FIG. 7a.

The borings thereby have a longitudinal cross-section as can be detected in FIG. 7a in plan view. They lead to conveyance of liquid from the base 29 of the agitation system 21 and hence of the mixing vessel 9 upwardly and therefore contribute to the formation of the liquid surface as a water spout.

FIG. 8 likewise shows a agitation system as was already illustrated similarly in FIG. 3. FIG. 8 thereby shows a cross-section, FIG. 8a a cross-section along the section line A-A in FIG. 8b and FIG. 8c a cross-section along the section line B-B in FIG. 8b. In contrast to FIG. 3, now there are however not two borings 34a and 34b which extend vertically from the surface of the agitation system into the interior thereof but in total four borings 34a to 34d which are offset relative to each other by an angle of 90°.

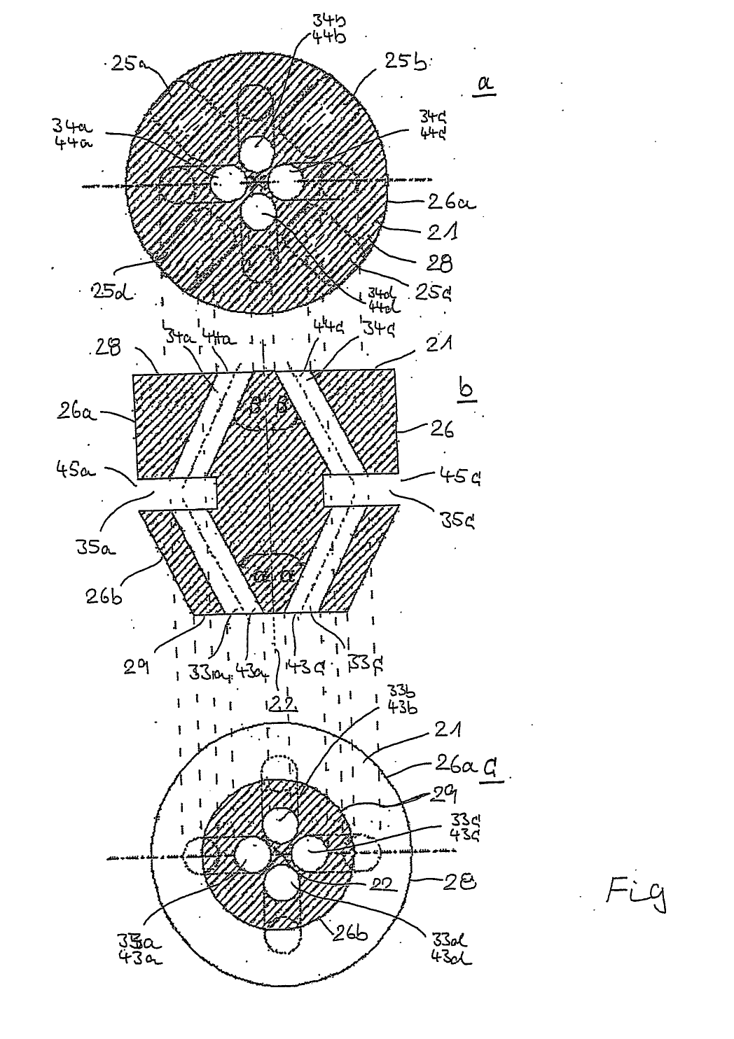

In FIG. 9, a further agitation system according to the invention is illustrated, FIGS. 9a and 9c representing a plan view or a bottom view of the agitation system illustrated in cross-section in FIG. 9b. Here also, four borings 34a to 34d which start from the upper side 28 of the agitation system 21 are disposed again in the impeller 21, offset about the central axis 22 of the agitation system 21 by 90°, these borings 34a to 34d now extending at an angle β to the central axis or rotational axis 22 of the agitation system 21 downwardly and outwardly. In the same, almost symmetrical manner, borings 33a to 33d extend from the underside 29 of the agitation system 21 at an angle a relative to the central axis or rotational axis 22 of the agitation system 21 upwardly and outwardly into the body of the agitation system 21. Both meet in total four horizontal channels 35a to 35d which are disposed offset relative to each other likewise by 90° and end with opening 45a to 45d in the side face 26 of the agitation system.

The side face 26 of this agitation system extends from its surface 28 perpendicularly downwards to the plane of the horizontal borings 35a to 35d and merges then conically parallel to the borings 33a to 33d downwardly.

The borings 33a to 33d in turn convey liquid from the base of a vessel upwardly whilst the borings 34a to 34d draw in gas and liquid or a mixture thereof from the surface 28 of the agitation system 21 and produce turbulences at the interface of gas and liquid. As a result, a pulsating Bernoulli effect is also generated or possibly increased.

A further possibility for generating a pulsating Bernoulli effect resides in using a non-rotationally symmetrical agitation system.

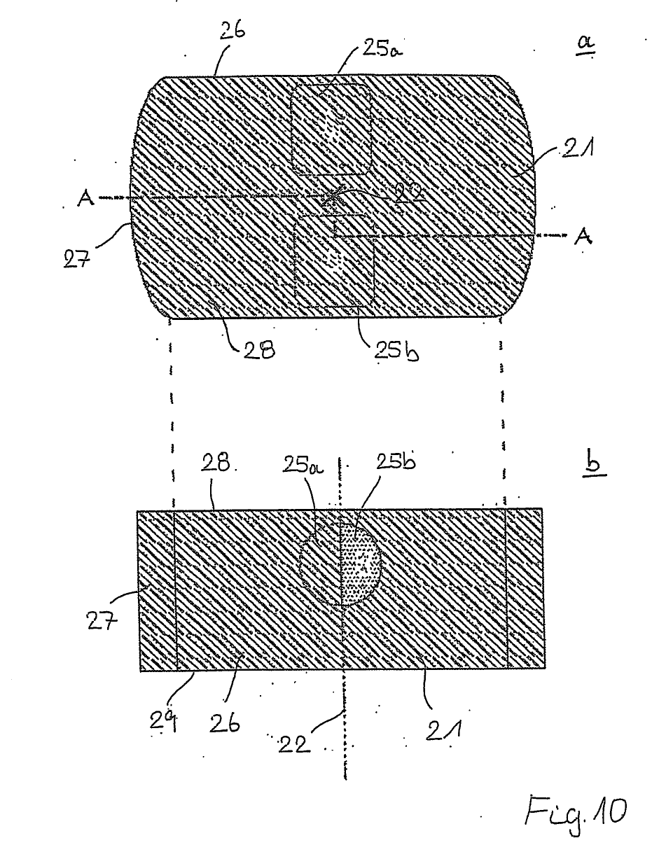

In the case of the agitation system 21 illustrated in FIG. 10, a linear front side 26 and a linear rear side 27 which is parallel to the front side 26 is present whilst the side faces 27 which go along the wall of the mixing vessel 9 and connect the front side 26 and the rear side 27 are curved convexly.

An agitation system 21 of this type can be used for example in a non-rotationally symmetrical mixing vessel or in a mixing vessel with baffles.

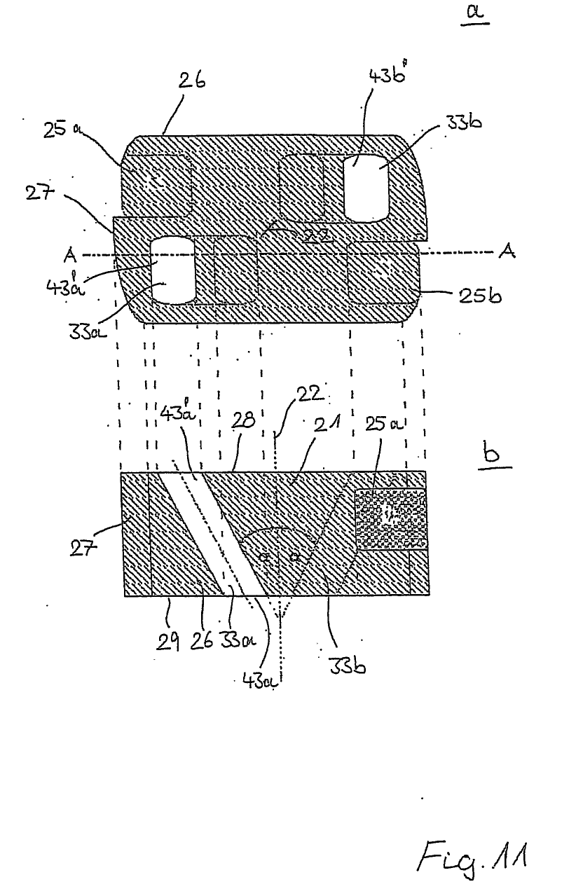

In FIG. 11, an agitation system as in FIG. 10 is illustrated, which in addition has borings 33a or 33b which extend at an angle a relative to the rotational axis 22. These convey liquid from the openings 43a, 43b on the underside 29 of the agitation system 21 to the openings 43a′ and 43b′ on the upper side 28 of the agitation system 21.

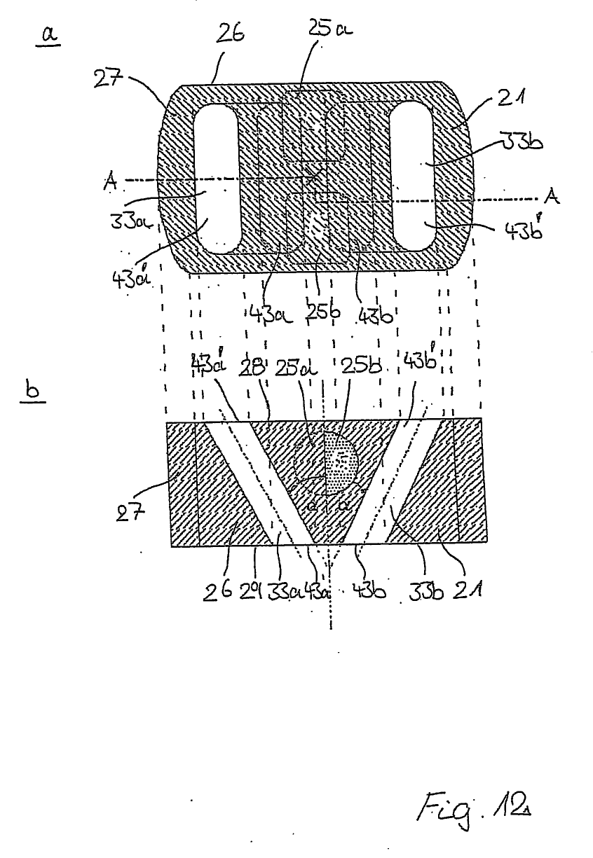

FIG. 12 shows a agitation system as in FIG. 11, now the borings 33a and 33b having a shape which can be detected in plan view in FIG. 12a and is elongated in cross-section.

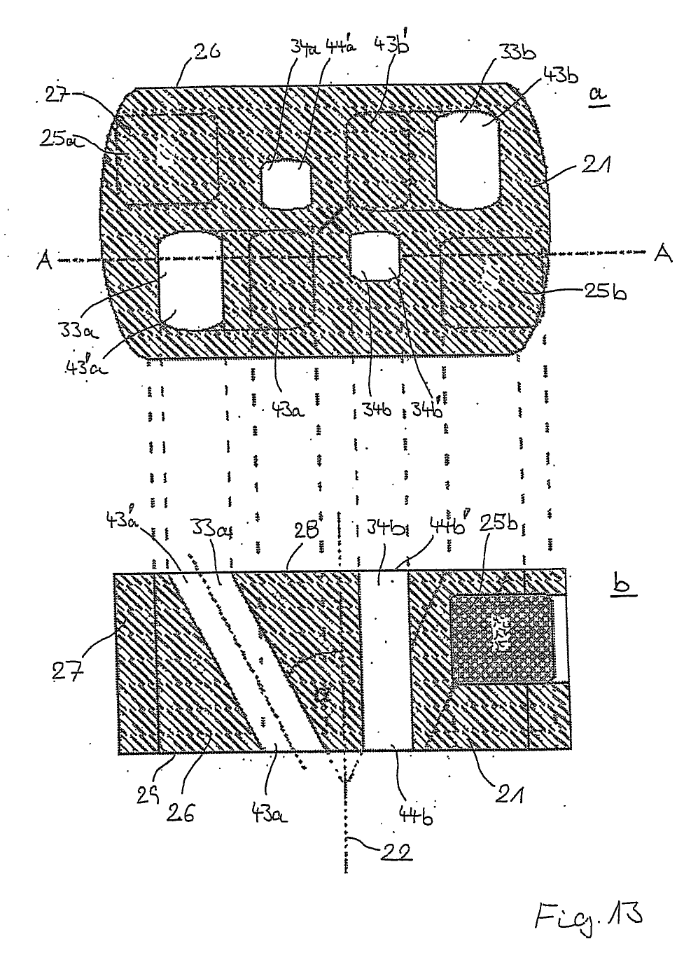

In FIG. 13, in addition to the borings 33a and 33b as in FIG. 11, further borings 34a, 34b are introduced which extend offset relative to the central axis 22 of the agitation system 21 parallel to the central axis 22 from the underside 29 to the upper side 28 and end respectively in openings 44a, 44b on the underside 29 or in openings 44a′, 44b′ on the upper side 28. These channels 34a, 34b serve for degassing the liquid, i.e. removing the spent gas bubbles which are dispersed in the liquid.

FIG. 14 shows in partial image a a reactor device according to the invention in which containers 9a to 9e are sunk in openings 8a to 8e in a reactor block 2. These containers 9a to 9e have in turn a flange on their upper circumferential edge of their opening, with which flange they are supported on a distance disc 11a to 11f. The entire arrangement of the containers is covered by a cover-like closure 15, in which, as already described above, tubes 17a to 17e are sunk in order to make it possible for gas to flow out. The cover 15 has furthermore separating walls 14a to 14e in order to isolate the individual containers from each other in a gas-impermeable manner.

FIG. 14b now shows a cross-section along the line A-A in FIG. 14a through the closure 15, whilst FIG. 14a represents a cross-section through the entire arrangement along the line B-B in FIG. 14b.

Starting from a gas supply boring 50, this boring branches via individual further borings 51, 52, 53, 54 to 55 continuously and finally discharges in respectively one gas outlet above the individual containers 9a to 9e. In FIG. 14b it can also be detected that the containers 9 are disposed in a two-dimensional array. The length of the borings 50 to 55 from the gas inlet 50 to the outlet in the air space above the vessels 9 is thereby respectively constant. Also the number of bends, which the individual gas conduits run through from inlet to outlet above the containers, is constant. The height of the channels can however thereby be variable. It is now crucial that no significant pressure drop takes place either in these borings 50 to 55 or that the pressure drop is identical for each individual container 9 due to the same length and the same number of bends.

FIG. 15 shows a further closure 15 with a corresponding gas distributor structure comprising channels 50 to 55, 51′ to 55′. Here also, the routes and the number of bends from the inlet 50 to the respective container 9 are identical for each container.

Now individual measurements with reaction vessels according to the invention or with a bioreactor block according to the invention are represented subsequently.

If the reactor block 1 is configured correspondingly geometrically (spacing and arrangement of the individual millilitre stirred tank reactors, e.g. respectively 8 millilitre stirred tank reactors in parallel—since pipetting robots are generally equipped with 8 parallel dosage stretches—etc.), simple automation can be effected. By means of a pipetting robot as actuator,
samples can be drawn individually and in principle all manual off-line analysis methods can be automated (determination of cell, substrate, product and byproduct concentrations),
correction means, substrate and/or inductors can be added individually,
the individual millilitre stirred tank reactors can be sampled intermittently with sensors (e.g. correspondingly dimensioned pH electrodes).

By means of suitable process control systems, an automated process control is hence possible in the parallel batch; pH adjustment, individual substrate dosage, automated off-line sampling and analysis and the like.

In principle, the following operational steps are necessary for automated implementation of parallel cell cultivations. Time planning is designed thereby such that the operational steps can be implemented in the course of the entire process in the same time cycle, i.e. maintenance of a constant timespan ΔT between the operational steps is made possible. Such a timeplan is represented in FIG. 17.
dispensing D: the millilitre stirred tank reactors operated in parallel are supplied by a pipetting robot with sterile, distilled water for evaporation control and/or with fresh medium to produce inflow or continuous methods and/or titration means for pH control and/or induction means. The respectively required volumes of each solution are calculated by algorithms which are initialised in advance with all the necessary parameters.
sampling: in order to be able to implement at-line analysis in an automated manner, prescribed sample volumes are removed regularly from the millilitre agitation reactors and placed in microtitre plates.
microtitre plate movements MTP Trans: after removing a prescribed number of samples into a microtitre plate, for example one sample per millilitre stirred tank reactor, the microtitre plate is transported by the laboratory robot to a microtitre plate measuring device.
at-line analysis: the bioreactor samples placed in a microtitre plate are measured in a measuring device (e.g. microtitre plate-photometer/fluorimeter). For example, the dry cell mass concentration (Cₓ) and the pH value can be determined thus. Also other bio-technically relevant parameters (e.g. substrate concentration, product concentration) can be determined in this way. The obtained measurement values are made available to control algorithms and thus have an effect on the following dispensing steps to be implemented by the laboratory robot. By establishing a cycle at least of two microtitre plates, the analysis can be effected in parallel to the dispensing and sampling steps implemented by the pipetting robot. A microtitre plate is accordingly filled with samples whilst the samples in the second microtitre plate are analysed in the measuring device. After conclusion of the analysis of the microtitre plate, the latter is cleaned in a microtitre plate washing appliance and supplied again to the cycle.
cleaning of the microtitre plate (“plate wash”): after completion of the test, the respective microtitre plate is cleaned and can thus be reused.

FIG. 16 shows an arrangement with microtitre plates 60a, 60b, 60c in a pipetting robot, the cycle of a sample microtitre plate 60a to 60c being able to be followed in this sketch. The automatic unit 56 illustrated in FIG. 16 has a base plate 57 on which an arrangement 64 of containers (reactor block) or sampling vessels has a table 58 for receiving microtitre plates 60a, a photometer 62 and a washing station 63. Furthermore, a carrier beam 66 is disposed above the base plate 57 on which beam the pipetting tips 65 and a bearing arm 61 for microtitre plates is suspended displaceably by means of transverse beams 59a and 59b.

In a first step, a microtitre plate 60a with samples from the reactor block 64 is now filled via the pipette tips 65. The microtitre plate 60a is then transported by the bearing arm 61 to the photometer 62 where the individual cups of the microtitre plates 60b are measured photometrically. The thus measured microtitre plate 60b is transported by the bearing arm to the washing device 63 where the microtitre plate (now microtitre plate 60c here) is washed and cleaned. Hence, the microtitre plate 60c is again available for samples and the measurement cycle and is conveyed by the bearing arm 61 back to the table 58.

According to the process requirements, microtitre plates filled with samples can be withdrawn from the process cycle at specific times and be cooled and stored in the interim. A new microtitre plate can be supplied automatically to the process in order to be able to maintain the analysis cycle.

Determination of materials dissolved in the aqueous reaction medium, such as the hydrogen ion activity (pH), must be effected individually in each reaction vessel. Use of 48 or 96 individual pH sensors, for example sterilisable pH glass electrodes are not economical. Also the use of economical pH field-effect transistors (“disposable sensors”) is in practice not possible due to the additionally required standard reference electrodes and the lack of thermal stability (ability to be sterilised).

The number of necessary pH sensors for parallel reactors can in principle be reduced if a sensor can be used for a plurality of reaction vessels. One possibility for technical production is the integration of commercially available miniature pH electrodes in piercing cannulae, which are immersed intermittently into the individual millilitre agitation reactors by means of a pipetting robot. pH single-rod measurement sequences with an external diameter of 1 mm and a response time of ˜6 s are suitable for this purpose.

A further possibility is sterile removal of samples from the individual millilitre stirred tank reactors with cannulae and parallel measurement of the pH value in the samples with pH-sensitive microtitre plates. On the base of the cavities of these commercially available microtitre plates, a sensor spot is integrated in which two fluorophores are immobilised. These fluorophores can be read in a correspondingly equipped photometer-fluorimeter. The fluorescence properties of the indicator-fluorophore vary with pH value of the solution whilst the reference fluorophore produces a fluorescence signal which is independent of the pH value. The ratio of indicator signal to reference signal can be correlated to the pH value of the solution via a sigmoid function. Use of a reference fluorophore increases the measuring precision and the lifespan of the sensors since a decrease in signal intensity by “bleeding out” of the sensor (diffusion of the fluorophores in the measuring solutions) results in a smaller effect on the measuring signal.

The produced pH measurement data are read by the process control system and made available to control algorithms. These calculate the necessary volume of titration means per reactor vessel in order to maintain a desired pH reference value in the reactor vessel. The process control system calculates the dispensing steps taking into account the necessary dosage for pH control.

The hence possible high oxygen and energy supply into a culture medium is essential in the present invention. Hence measurements of the oxygen transfer coefficients k_{L}a of different magnetic agitation systems according to the invention are described subsequently.

Agitation systems of the Types I to V corresponding to FIG. 7 (Type I), FIG. 10 (Type II), FIG. 12 (Type III), FIG. 11 (Type IV) and FIG. 9 (Type V) were compared with the dynamic sulphite method (Havelka et al. 1998) under comparable conditions with respect to volume of the liquid phase, mounting of the agitation system and arrangement of the baffles (see FIG. 18). These tests were implemented in a millilitre stirred tank reactor with 15.5 mm diameter. With the impellers of Types I to V at a rotational speed of 2600 rpm, k_{L}a values in 0.5 M Na₂SO₄ solution in the range 0.156-0.244 s⁻¹ were achieved.

In order to determine the k_{L}a values, 0.5 M Na₂SO₄ solution is used, which guarantees non-coalescing conditions. In addition, a concentration of 10⁻³ M CoSO4 is present as catalyst for the chemical oxidation of sulphite into sulphate. Implementation of the dynamic sulphite method begins with aeration of the liquid phase with air until the latter achieves saturation. After addition of a sufficiently large material quantity of sulphite in order to consume all the oxygen dissolved in the liquid phase, the dissolved oxygen concentration of the liquid phase drops abruptly to zero. After stoichiometric sulphite conversion, the dissolved oxygen concentration in the liquid phase increases again. From this so-called reconcentration curve, the k_{L}a value is determined by assuming ideally mixed conditions in liquid and gas phase. The response time of the used oxygen probe in the model is thereby taken into account.

Conventional technical stirred tank reactors are operated with oxygen transfer coefficients of k_{L}a <0.25 s⁻¹. Hence, in millilitre stirred tank reactors according to the invention or with the agitation system according to the invention, the same or similar oxygen transfer rates can be achieved (by way of comparison: in agitated flasks or microtitre plates, oxygen transfer coefficients of at most k_{L}a=0.07 s⁻¹ can be achieved under optimal conditions).

Oxygen transfer coefficients of the agitation systems of Type III and V were furthermore implemented with 8 ml 0.5 M Na₂SO₄ solution likewise in a millilitre agitation reactor with 20 mm diameter (FIG. 19). Agitation systems according to FIG. 2b and 2c were thereby used. Maximum achieved k_{L}a values for a rotational speed of 2800 rpm are here even up to 0.355 s⁻¹.

In order to verify the oxygen transfer properties of the agitation systems, parallel cultivations with Escherichia coli (wt) were implemented as example system.

100 ml of a sterile, defined medium were inoculated with 0.25% inoculum from the feedstock in a 500 m agitated flask sealed with Alucap and incubated for 14 h at 37° C. and 200 rpm in an agitated incubator with an eccentricity of 5 cm. On the following day, 4-6 ml of this preculture were transferred into millilitre agitation reactors. In the millilitre agitation reactors, the cells were incubated for 3.5 h at 2000 rpm of an agitation system according to the invention and for a further 2.5 h at 2200 rpm at 37° C. The pH value was controlled to 6.8.

The agitated flask (AF) was incubated further with the approx. 40 ml residual volumes of preculture in the agitated incubator under the same conditions.

The measurement results in FIG. 20 to 22 prove that a further growth of the bacteria is possible in the millilitre agitation reactors if sufficient oxygen is introduced. The respective curves thereby show a conventional agitated flask (AF), a mixer of a specific type according to the invention without baffles (e.g. “Type II without FB”) or with baffles (e.g. “Type II”). It can be detected from FIGS. 20 to 22 that a rotational speed of 2200 rpm already made possible, in the batches with the agitation systems of Type II, III and V, an up to 2.5 times higher dry cell mass concentration than possible in the agitated flask batch (AF).

FIG. 23 now indicates respectively in Table form in what configuration the individual agitation systems were used. For the measurements which are presented in a specific Figure, the vessel diameter and the number of baffles is thereby indicated respectively. Furthermore, the configuration of baffles and agitation system is indicated with reference to the representation in FIGS. 2a to 2d.

FIGS. 24 to 26 show further embodiments of agitation systems according to the invention. These agitation systems are all prepared for mounting on a shaft 23, in that borings 50 and 51 are introduced into the agitation system 21. In the upper region of the agitation system, the boring 51 has a clearance width so that it makes possible passage and mounting of the shaft 23. FIG. 24a now shows a plan view, the different diameters of the borings 50 and 51 being illustrated. FIG. 24b shows a cross-sectioal view of the same agitation system.

The borings 33a and 33b are now continuous from a lower edge of the agitation system to the oppositely situated upper edge of the agitation system. The borings 33a and 33b are thereby situated in a section plane so that they intersect in the region of the boring 50 and form a common cavity. The openings of the borings 33a and 33b now extend both over a part of the underside 29 or of the upper side 28 of the agitation system 21 and over its side wall. As a result, four partial borings, in a star-shape, thus extend towards each other in the centre from opposite sides on the upper and the lower edge of the agitation system.

This agitation system can be self-centring or also, as illustrated in FIG. 24, can be mounted on a shaft 23 introduced into the reaction vessel. The shaft 23 can thereby be mounted for example on the cover of the reaction vessel. In FIG. 24, the shaft 23 has an enlargement or a flange 50 at its lower end situated in the boring 50 so that the agitation system cannot fall from the shaft 23 since the boring 50 in the upper region narrows continuously into the boring 51. This enlargement 55 can likewise be configured as a key face. The shaft, which now, deviating from the preceding embodiments, protrudes from above into the reaction vessel, and is immersed in the liquid phase, can be configured as solid material as in FIG. 24. Exact positioning of the shaft end relative to the internal geometry of the agitation system is thereby essential in order to achieve an effective suction effect and hence to intensify the introduction of bubbles.

FIG. 25 shows a similar agitation system as in FIG. 24, with the difference that the outer form of the agitation system, as can be detected in the cross-section in FIG. 25a, is in principle circular. This agitation system now has in addition recesses 56a to 56b which, during circulation in the flow, effect a temporal or spatial change in the flow velocity along the circumference of the agitation system 21. As a result, even better mixing and introduction of bubbles is achieved. Furthermore, as can be detected in the lateral cross-section of FIG. 25b, the shaft 23 is configured as a hollow pipe so that gas conveyance from above to below into the liquid phase takes place via the central cavity 57.

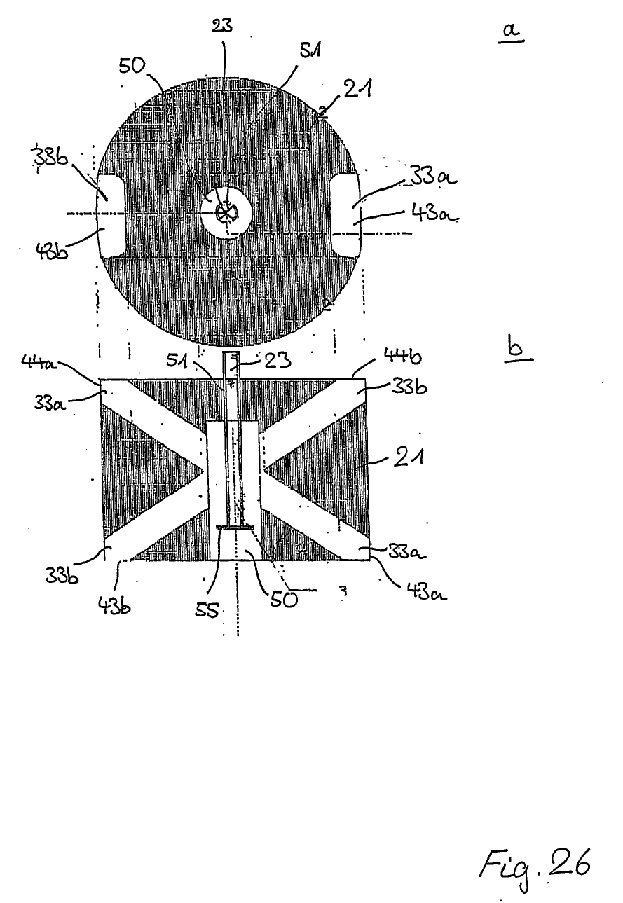

FIG. 26 shows a corresponding agitation system as in FIG. 25, however no recesses 56a to 56b being provided and the shaft 23 comprising solid material as in FIG. 24.

FIG. 27 shows now in the partial Figures a) and b) both variants of the mounting of the agitation systems with shafts 23 which are immersed from above into the liquid phase 30. The shaft 23 in FIG. 27a is configured as a hollow pipe so that, corresponding to arrows A and A′, a gas feed takes place into the liquid phase. In FIG. 27b, the shaft 23 is configured as solid material so that it serves merely for mounting and if necessary rotating the agitation system 21.

FIG. 28 shows a device according to FIG. 27a, however a nozzle 60 being disposed at the lower end of the shaft 23 for further intensification of the oxygen feed and bubble formation in the liquid phase 30. As shown in FIG. 28, this can either be disposed rigidly on the shaft 23 or possibly serve also simultaneously as stop member (flange) for the agitation system 21. Furthermore, it can be pressed also into the rotating agitation system 21 instead of being disposed on the shaft 23. The nozzle 60 contains an air transfer boring 58 which connects the cavity 56 of the shaft 23 to the head of the cone of the nozzle 60. From this head of the nozzle cone 60, a plurality of outlet borings 59a and 59b leads upwards or in the direction of the agitation system 21, air being fed into the liquid phase 30 in the direction of the arrows B and B′. Here also an exact orientation relative to the agitation system is important for a high oxygen transfer.

FIG. 29 shows the maximum oxygen transfer coefficient which can be achieved with an agitation system according to FIG. 25 with 8 or 12 ml reaction volume. In particular with a reaction volume of only 8 ml, an oxygen transfer coefficient of 0.4 s⁻¹ can already be achieved with a rotational speed of 2300 rpm.

FIG. 30 shows the growth of the dry cell weight concentration (BTM) in a fed-batch cultivation with Escherichia coli wt K12. With feeding, a bio dry mass concentration of above 13 gL⁻¹ was achieved within 9 hours. The rotational speeds of the agitation system, which corresponded to the one in FIG. 25, were thereby not above 2200 rpm, which nevertheless ensured an oxygen concentration of the medium of above 25% air saturation.

The cultivation was effected here in a mineral medium with 15 gL⁻¹ glucose at the beginning of the batch phase as starter concentration. After consumption of this glucose after 4.15 h, a glucose solution with 250 gL⁻¹ glucose content was fed intermittently every 4 minutes. The pH value was set to 6.8 by means of 2.5% NH₄OH.

In summary, it can be consequently established that parallel millilitre stirred tank reactors have been made possible by the present invention, in which comparable growth as in controlled processes in bioreactors of a laboratory scale is made possible.

Surface-aerated millilitre agitation reactors equipped if necessary with special magnetic agitation systems permit an equally efficient oxygen supply for organisms in liquid culture as mixing vessel reactors of a greater scale with volume aeration.

The use of up to 96 and more millilitre stirred tank reactors in one bioreactor block which can be automated by means of pipetting robots makes possible for the first time an efficient, individually controlled parallel operation. The use of a laboratory robot for automation of the parallel reactions hence makes possible a quantum leap in obtaining relevant process data.

By implementing suitable screening and optimising routines, screening methods and process optimisations (media composition, induction methods, dosage profiles) can be automated systematically and with high time efficiency in the parallel batch. Complete digitalisation of the parallel process development makes further possible a novel data transparency and availability.

By means of this new tool for high-throughput bioprocess development, new bioprocesses can be developed in a time-efficient manner under technical reaction conditions since, for example instead of an experiment in the controlled 0.5 l mixing vessel reactor with the same reaction volume, 100 experiments can be implemented at the same time in 100 parallel 5 ml mixing vessel reactors in an automated manner—i.e. an information yield per unit of time, at a multiple of 100, becomes possible. When using suitable test planning algorithms, the effectiveness of the bioprocess development is further increased.

1. Device for cultivating cells in liquid columns on a millilitre scale, having a container for receiving a liquid culture suspension of the cells, an agitation system for mixing the culture suspension in the container about a mixing axis, characterised in that at least one of the container and the agitation system are configured in such a manner that the flow velocity is modified at least one of locally and temporally along a streamline or flow line about the agitation axis.

2. Device according to claim 1, characterised in that at least one of the container and the agitation system are configured in such a manner that, by means of the agitation system, conveyance of the culture suspension which is directed from the suspension surface to the base of the container is effected.

3. Device according to claim 2, characterised in that the conveyance of the culture suspension has a component which is axial with respect to the mixing axis.

4. Device according to claim 1, characterised in that the inner walls of the container which enclose the culture suspension have, below, at least one of and above the rotational plane of the agitation system, a non-rotationally symmetrical form.

5. Device according to claim 1, characterised in that the inner walls of the container which enclose the culture suspension form, below at least one of in and above the rotational plane of the agitation system, a polygon, preferably with four, five, six or more corners.

6. Device according to claim 1, characterised in that the rotational axis of the agitation system is disposed off-centre or eccentrically in the container with respect to the inner walls of the container which enclose the culture suspension.

7. Device according to claim 1, characterised in that at least one baffle is disposed on the inner wall of the container along the circumference of the agitation system.

8. Device according to claim 1, characterised in that one, two, three, four or more baffles are disposed along the inner wall of the container, at a spacing, advantageously at a uniform spacing, relative to each other.

9. Device according to claim 7, characterised in that the baffle or baffles is or are disposed at least one of below, in and above the rotational plane of the agitation system.

10. Device according to claim 7, characterised in that the agitation system and the baffle are disposed at a minimum gap spacing relative to each other of 0.05 mm to 20 mm, preferably 0.1 mm to 3 mm.

11. Device according to claim 1, characterised in that the container is a flask, a reagent glass or a cavity of a microtitre plate or of another plate which is provided with cavities.

12. Device according to claim 1, characterised in that the agitation system is at least one of magnetically positioned and driveable.

13. Device according to claim 1, characterised in that the agitation system is mounted or not mounted.

14. Device according to claim 7, characterised in that the agitation system is mounted via a shaft and, if necessary, is driveable via the shaft.

15. Device according to claim 14, characterised in that the shaft and the baffle or baffles are configured in one piece.

16. Device according to claim 14, characterised in that the shaft and the baffle or baffles is or are insertable into the container.

17. Device according to claim 14, characterised in that the shaft protrudes into the container from above.

18. Device according to claim 14, characterised in that the shaft is enlarged at its lower end or has a flange.

19. Device according to claim 14, characterised in that the shaft is configured as a solid material shaft or as a hollow pipe.

20. Device according to claim 14, characterised in that the shaft is configured as a hollow pipe and a nozzle is disposed on its end which is open at the bottom.

21. Agitation system having a basic body which, in operation, has an upper side and an underside, characterised in that, in the basic body, at least one first through-channel is disposed, the first opening of which is situated at least partially on the underside of the basic body and the second opening of which is situated on at least one of the upper side and laterally on the basic body.

22. Agitation system according to claim 21, characterised in that, in the basic body, at least one first boring is disposed as first through-channel, the passage axis of which boring includes an angle α with 0°≦α<90° with a rotational axis of the agitation system, said angle opening to the upper side of the agitation system.

23. Agitation system according to claim 21, characterised in that, in the basic body, at least one further second through-channel is disposed, the first opening of which is situated at least partially on the upper side of the basic body and the second opening of which is situated on at least one of the underside and laterally on the basic body.

24. Agitation system according to claim 23, characterised in that, in the basic body, at least one second boring is disposed as second through-channel, the passage axis of which boring includes an angle α with 0°≦α<90° with the rotational axis of the agitation system, said angle opening to the underside of the agitation system.

25. Agitation system according to claim 23, characterised in that at least one of the first and the second through-channel intersect and the first and the second through-channel meet each other and, forming a common through-channel, emerge laterally from the agitation system.

26. Agitation system according to claim 23, characterised in that two or more of the at least one of the first and second through-channels are disposed along the circumference of the agitation system at a uniform spacing relative to each other.

27. Agitation system according to claim 22, characterised in that the basic body, in the cross-section perpendicular to the rotational axis, is circular cylindrical, elliptical, polygonal, square or rectangular.

28. Agitation system according to claim 21, characterised in that the basic body is oval, egg-shaped or cuboid.

29. Agitation system according to claim 21, characterised in that the basic body has recesses along its circumferential edge in the rotational direction.

30. Agitation system according to claim 1, characterised in that the agitation system has a basic body which, in operation, has an upper side and an underside wherein in the basic body, at least one first through-channel is disposed, the first opening of which is situated at least partially on the underside of the basic body and the second opening of which is situated on at least one of the upper side and laterally on the basic body.

31. Arrangement for parallel, automated cultivation of cells in liquid columns on a millilitre scale, characterised by at least one device or agitation system according claim 1.

32. Arrangement according to claim 31, characterised by a block, in which a number of cavities, which corresponds to the number of containers, are disposed, which represent containers themselves or are configured for receiving containers.

33. Arrangement according to claim 32, characterised in that the cavities are configured as borings with a diameter corresponding to the external diameter of the containers.

34. Arrangement according to claim 32, characterised in that at least one of means for moderating the temperature of the block, means for driving the magnetic agitation system and a sterile gas supply to the containers are disposed in the block.

35. Arrangement according to claim 30, characterised in that at least one of the containers, the arrangement and the block are sealed in a sterile manner on their or its upper side by means of a cover.

36. Arrangement according to claim 35, characterised in that the cover has an opening for the release of gases and as access to the interior of the container or block, which opening extends in a straight line and longitudinally from the interior to the outer side of the cover and connects these together in an open manner for gas convection.

37. Arrangement according to claim 36, characterised in that, in the interior, a higher gas pressure is present than on the outer side of the cover.

38. Arrangement according to claim 36, characterised in that the opening has the form of a boring or of a tube.

39. Arrangement according to claim 36, characterised in that the opening has a clearance width, which makes possible the introduction of a sampling element or of a sensor, a pipette tip, a piercing cannula, a pH electrode or another object or elongated object into the interior of the container or block.

40. Arrangement according to claim 36, characterised in that the opening is a tube made of metal or metal alloys, advantageously made of at least one of aluminum and silver.

41. Arrangement according to claim 36, characterised by container separating elements which, when in the state set upon the containers or the block, separate individual containers from each other in at least one of a gas- and liquid-impermeable manner.

42. Arrangement according to claim 36, characterised in that the cover has a sterile gas supply to the one or a plurality of containers, for all the containers together or for a plurality or each of the containers separately.

43. Arrangement according to claim 42, characterised in that the sterile gas supply has gas distributor structures which are integrated in the cover or disposed adjacent to the cover.

44. Arrangement according to claim 43, characterised in that channels are disposed in the cover as gas distributor structures.

45. Arrangement according to claim 44, characterised in that the channels between the gas inlet into the gas distributor structure and the gas outlets into the respective containers all have at least one of the same length and the same number of bends.

46. Arrangement according to claim 42, characterised in that the gas supply is connected to a gas feed, if necessary via at least one of sterile filters and air humidifiers.

47. Arrangement according to claim 31, characterised in that the cover has at least one planar layer or flat plate, which covers the opening of the at least one container.

48. Arrangement according to the claim 47, characterised in that it has at least two planar layers which are disposed parallel to each other and between which the gas distributor structures are disposed.

49. Method for cultivating cells in liquid columns on a millilitre scale, at least one cell suspension being moved in a container in such a manner that the cell suspension forms an annular flow, characterised in that the flow velocity of the annular flow is modified at least one of locally and temporally along a streamline.

50. Method according to claim 49, characterised in that at least one of the container and the agitation system are configured in such a manner that conveyance of the culture suspension which is directed from the suspension surface to the base of the container is effected.

51. Method according to claim 49, characterised in that the at least one cell suspension is cultivated in a device or arrangement according to one of the preceding claims by mixing with an agitation system.

52. Method according to claim 51, characterised in that the agitation system is driven with 1 to 4000 revolutions per minute.

53. Method according to claim 51, characterised in that the agitation system is driven with over 500 revolutions per minute.

54. Method according to the claim 53, characterised in that the agitation system is driven with over 1000 revolutions per minute.
